(19) United States
(12) Patent Application Publication
(10) Pub. No.: US 2024/0389585 A1
(43) Pub. Date: Nov. 28, 2024

(54) METHOD FOR PROMOTING PLANT GROWTH

(71) Applicants: Nanyang Technological University, Singapore (SG); National University of Singapore, Singapore (SG)

(72) Inventors: Sanjay SWARUP, Singapore (SG); Omkar Shashikant KULKARNI, Singapore (SG); Mrinmoy MAZUMDER, Singapore (SG)

(21) Appl. No.: 18/722,988

(22) PCT Filed: Dec. 22, 2022
(86) PCT No.: PCT/SG2022/050926

§ 371 Date: Jun. 21, 2024

(30) Foreign Application Priority Data
Dec. 23, 2021 (SG) 10202114308S

(51) Int. Cl. A01N 37/42; A01P 21/00; C05F 11/08; C12N 1/20

(52) U.S. Cl. CPC A01N 37/42; A01P 21/00; C05F 11/08; C12N 1/20

(57) ABSTRACT

This disclosure concerns plant-derived organic compounds and their use thereof to promote the growth of plants and biofilms.

### TECHNICAL FIELD

The present invention relates, in general terms, to plant-derived organic compounds and their use thereof to promote the growth of plants and biofilms.

### BACKGROUND

As the world population is projected to reach 10 billion by 2050, food security has become one of the largest challenges of the 21st century. Agricultural productivity must be met sustainably despite the backdrop of climate change, land degradation, and increasingly unpredictable weather events. The use of agricultural microbials (or agro-microbials) and nature-based agrochemicals are now widely considered a promising strategy for sustainable agriculture.

Agro-microbials encompass crop-associated microbial communities that provide indispensable functions, including plant growth promotion, disease prevention, and nitrogen fixation. They also keep the soil fertile by breaking down organic matter, recycling nutrients, and creating humus to retain moisture. Rather than being free-floating or planktonic, a large percentage of soil microbes adopt the biofilm mode of life, where they are attached to a surface and embedded in a self-secreted polymeric matrix. This lifestyle confers various advantages to the microbial members, such as adhesion/cohesion capabilities and protection from environmental stressors. The diverse microbial community in biofilms may communicate with each other and share available resources.

Microbes can be 100-fold more abundant in vegetated compared to non-vegetated soils, and the majority colonise and form biofilms in the region around plant roots known as the rhizosphere. The rhizosphere microbiome can be distinct from the bulk soil microbiome, and is shaped by compounds released by the plants, including root exudates and volatile organic compounds (VOCs). Soluble root exudate components like coumarins, benzoxazinoids, salicylic acid, flavones, fumaric acid and citric acid establish a zone of rhizospheric influence that extends 2 to 10 millimetres from the root surface, to which soil microbes are drawn and within which they assemble into biofilms. These biofilms in turn provide beneficial feedback to the plant host, including nutritional provisioning through nitrogen fixation, direct protection against pathogens and indirect protection through induction of stress tolerance. The ability of root-derived compounds to influence biofilm assembly is largely unknown.

Accordingly, it would be desirable to overcome or ameliorate at least one of the above-described problems.

### SUMMARY

The present disclosure provides a method for promoting plant growth in a growth medium, the method comprising inducing biofilm growth in the growth medium by providing an oxylipin to the growth medium, wherein induction of biofilm growth promotes plant growth in the growth medium.

Disclosed herein is a method for inducing biofilm growth in a growth medium, the method comprising providing an oxylipin to the growth medium, wherein the root volatile organic compound induces biofilm growth in the growth medium.

Disclosed herein is a growth medium for promoting plant growth, wherein the growth medium comprises an oxylipin.

Disclosed herein is a kit for promoting plant growth, wherein the kit comprises a growth medium, wherein the growth medium comprises an oxylipin.

Disclosed herein is a method for isolating a microbe that is responsive to an oxylipin, the method comprising a) culturing a plurality of microbes in a growth medium comprising the oxylipin; and b) isolating microbes that are enriched in the growth medium comprising the oxylipin as compared to a growth medium that does not contain the oxylipin.

### BRIEF DESCRIPTION OF THE DRAWINGS

Embodiments of the present invention will now be described, by way of non-limiting example, with reference to the drawings in which:

FIG. 1 shows that plant root VOCs promote biofilms in complex soil microbiota. A) Schematic showing the preparation of soil microbiota inoculum; B) Schematic showing the static system for assaying the effect of plant VOCs on microbiota; C) Biofilm growth quantification in the static system using CV staining, following exposure to volatiles from Arabidopsis thaliana (each dot represents a biological replicate, p-values calculated using t-test); D) Dynamic push-pull system to assay the effect of plant root volatiles (rVOCs) on soil microbiota in the recipient chamber, wherein (C): Charcoal filter, (M): Microbial filter, (G): Gas wash bottle, (SC): Source Chamber, (RC): Receiving Chamber, (MI): Microbiota Inoculum; E) Biofilm biomass quantification in the dynamic system using CV staining, following exposure to rVOCs from Arabidopsis thaliana (each push-pull setup is treated as one biological replicate in pair, each dot represents a biological replicate which is an average of 4 technical replicates, p-values calculated using paired t-test); F) Biofilm quantification with and without rVOCs from a variety of species (each push-pull setup is treated as one biological replicate in pair, each dot represents a biological replicate which is an average of 4 technical replicates, error bars indicate +/−standard error, p-values calculated using paired t-test) (*) signifies p<0.05, (·) signifies p<0.1.

FIG. 2 shows that oxylipins are the major class of root-VOCs involved in biofilm promotion. A) Major volatile biosynthetic pathways in Arabidopsis and their selected biosynthetic mutant gene names, wherein ggpps: Geranyl Geranyl Pyrophosphate Lyase, tps4: terpene synthase 4, tgg 1/2: beta-thioglucoside glucohydrolase-1/2, cyp83a1: cytochrome p450, family 83, subfamily a, polypeptide 1, hpl: hydroperoxide lyase, lox1: lipoxygenase-1, pal1: Phenylalanine ammonialyase1, fps1 Farnesyl pyrophosphate synthase-1, jmt: jasmonate methyltransferase; B) Crystal violet staining assay performed to screen biosynthetic mutants to detect their lack/presence of biofilm-promoting ability in the static system (p-values calculated using t-test after performing pairwise comparisons of wildtype rVOCs biofilms to that of individual mutants); C) Crystal violet staining assay performed on selective mutants in dynamic system (each push-pull setup is treated as one biological replicate in pair, each dot represents a biological replicate which is an average of 4 technical replicates, error bars indicate +/−standard error, p-values calculated using paired t-test) (**) signifies p<0.01, (*) signifies p<0.05, (·) signifies p<0.1.

FIG. 3 shows that methyl jasmonate (MeJA) in root volatiles modulates biofilms in complex soil microbiota. A) Extracted ion chromatogram for ions belonging to methyl jasmonate (83, 151, 224) obtained from TD-GCMS of VOCs from the soil, jmt roots, and WT roots. B) Mass spectra of MeJA detected from samples (top panel) and NIST library (Bottom panel). C) Methyl jasmonate quantifier ion (83) intensity in VOCs captured from the soil, roots of jmt, and WT Arabidopsis (each dot represents a biological replicate, p-values calculated using Wilcoxon test). D) Quantification of biofilms formed after treatment of different concentrations of MeJA in the push-pull dynamic system using crystal violet staining assay (boxplot and p-values are based on 4 biological replicates whereas the gray dots indicate technical replicates. p-value calculated from paired t-test) (*) signifies p<0.05, (·) signifies p<0.1. E) Representative snapshots of soil microbiota biofilms (nucleic acids) stained with SYTO9 treated with (right panel) and without (left panel) 5 nM of MeJA. F) Representative snapshots of soil microbiota biofilms (extracellular matrix proteins) stained with SYPRO RUBY treated with (right panel) and without (left panel) 5 nM of MeJA. G) Quantification of 3D biovolume of biofilm members shown in panel E, H) Quantification of 3D biovolume of biofilm matrix shown in panel H, line smoothing performed using generalized linear model, faded region represents 95% confidence interval, line smoothing performed using generalized linear model, the faded region represents 95% confidence interval.

FIG. 4 shows that phylogenetically diverse strains respond differentially to host VOCs. A) Experimental design for identification of rVOC- and MeJA-responders. Boxes represent components of push-pull airflow system; B) Shannon diversity of all the samples in both phases (planktonic-biofilm) and the inocula; C-D) Comprehensive heatmap of rVOC and MeJA responders

FIG. 5 shows that host VOC-induced complex biofilms promote plant growth. A) Experimental setup to study the effect of Host VOC (plant rVOCs or pure MeJA)-induced biofilms on the plant growth. The two-step process consists of (1) generating complex biofilms of soil microbiome exposed to host VOCs over 24 hours, (2A) harvesting microbiota from planktonic and biofilm fractions separately, followed by co-culturing with 4-days old seedlings without any physical contact, (2B) harvesting of intact biofilms (no planktonic) followed by their co-inoculation with 4-days old seedlings without any physical contact. In both assays, microbiota/biofilms exposed to only soil VOCs was taken as a control. Plants were continuously monitored over 16 days to obtain their digital biomass. Representative images of phenotypes resulting from exposure to soil, root VOC-induced biofilm microbiome with shared headspace allowing gaseous exchange. B) Representative images of plant phenotypes resulting from co-culture with No-rVOCs- and rVOCs-exposed planktonic and biofilm community. C) Digital quantification of leaf and root area from the assay shown in panel B. P-values calculated using Wilcoxon rank sum test. D) Representative images of plant phenotypes resulting from co-culture with No-rVOCs and rVOCs-induced intact (microbiome+matrix) biofilms. E) Growth dynamics of leaf area (digital) of plants from the assay shown in panel D (n=4). F) Representative images of plant phenotypes resulting from exposure to No-MeJA (soil) and MeJA VOCs-induced intact (microbiome+matrix) biofilms. G) Growth dynamics of leaf area (digital) of plants from the assay shown in panel F (n=4). Error bars indicate standard errors for panel D and G. Linear mixed effect model was adopted (random effects: each replicate, fixed effects: time and treatments) to analyse the differences between control and host-VOC-biofilm-mediated plant growth. ‘*’ indicates p<0.05, ‘·’ Indicates p<0.1.

FIG. 6 shows that MeJA-responder strains recapitulate plant growth promotion. A) Phylogenetic tree of microbial isolates used for this study based on their 16S sequence. B) Biofilm biomass of microbial isolates in response to 5 nM MeJA. C-D) Effect of VOCs from microbial isolates on plant growth.

FIG. 7 shows the isolation of MeJA responders from soil biofilm and their effects on plant growth. A) Schematic representation of the total workflow to isolate and identify MeJA responders from biofilm. B) The list of isolated strains from soil biofilm adopting the above-mentioned workflow. C) The effects of some of the selected isolates on plant growth in an in vitro plate assay system.

FIG. 8 shows the quantification of biofilm biomass using crystal violet (CV) staining. Complex soil microbiome was treated with various MeJA chemical analogs over 20 hrs and stained with CV. The full chemical name of the chemical analogs are as follows: DHJ: Dihydro jasmonic acid; MDHJ: Methyl dihydro jasmonate; MJ: Methyl jasmonate; MO: Methyl octanoate.

FIG. 9 shows an effective dynamic range of liquid MeJA concentration for biofilm induction by soil microbiome.

FIG. 10 shows that MeJA-induced biofilms selectively trap pathogens. A) Schematic showing the experimental set-up. B) The amount of plant pathogen Xanthomonas sp. trapped in control and MeJA-induced biofilms. C) The amount of plant-beneficial strain P. protegens Pf-5 trapped in control and MeJA-induced biofilms.

FIG. 11 shows the biofilm lifestyle-related transcriptomic signature of P. protegens Pf-5 in response to total plant VOCs. A) Experiment design. The streaks in the right compartment of both plates represents 2 μL of 10⁴ CFU/mL of Pf-5. A 0.4 cm gap is created between these two compartments through the removal of MS agar; B) Statistically significant differentially abundant genes responding to total plant VOCs (adjusted p value<0.05)

FIG. 12 shows response of garden soil microbiota to Arabidopsis rVOCs. Related to FIG. 1. A) Microbiota was harvested from garden soil and biofilm assay was performed in the push-pull setup and biofilm was quantified using crystal violet staining assay (p-value calculated from paired t-tests).

FIG. 13 shows planktonic-biofilm fractionation of soil microbiota induced by rVOCs. A) Bacterial counts as measured by flow cytometry for 2 life phases (planktonic and biofilms) of the soil microbiome inoculumn exposed to plant rVOCs and soil VOCs.

FIG. 14 shows that the volatile sampling system specifically samples root volatiles. A) Extracted ion chromatogram for ions belonging to methyl jasmonate (83, 151, 224) from obtained from TD-GCMS of samples indicated in the left panel. White square indicates filter paper soaked with MeJA. B) Mass spectra of MeJA detected form samples (top panel) and from NIST library (bottom panel).

FIG. 15 shows that MeJA displays dose-dependent modulation of biofilm growth. A) Biofilm growth rate was calculated by normalizing the biovolume of the first frame of imaging and calculating growth compared to initial biovolume of respective samples.

FIG. 16 shows that complementation of jmt mutant by MeJA partially recovers ability to induce biofilms. A) Biofilm staining was performed at 16 and 24 hours. p-value calculated from paired t-test (*) signifies p<0.05, (·) signifies p<0.1 as calculated by paired-t test.

FIG. 17 shows the workflow for microbiome analysis of volatile-exposed communities. A) Flowchart for data analysis used for performing Quantitative Microbiome Profiling. B) Rarefaction curves showing that the read depth was sufficient to detect the existing taxa from biofilm and planktonic samples. C) Principal Coordinate Analysis (PCoA) performed by plotting bray-curtis distances. D) Pearson correlation between DNA yield and 16S copy numbers, E) Constrained analysis of principal coordinates using bray-curtis distance between planktonic and biofilm communities exposed to different VOCs.

FIG. 18 shows the predicted functions of biofilm community in response to MeJA exposure at 16 and 24 hours. A) Log2 fold change of gene abundances as calculated from predicted metagenomes based on 16S rRNA gene sequences.

### DETAILED DESCRIPTION

The present specification teaches a method for promoting plant growth in a growth medium, the method comprising inducing biofilm growth in the growth medium by providing a root volatile organic compound to the growth medium, wherein induction of biofilm growth promotes plant growth in the growth medium.

Disclosed herein is a method for promoting plant growth in a growth medium, the method comprising inducing biofilm growth in the growth medium by providing an oxylipin to the growth medium, wherein induction of biofilm growth promotes plant growth in the growth medium.

As used herein the terms “microorganism” and “microbe” should be taken broadly. These terms are used interchangeably and include, but are not limited to, the two prokaryotic domains, Bacteria and Archaea, as well as eukaryotic fungi, algae and protists. Any reference to an identified taxonomic genus should be taken to include identified taxonomic species in that genus, as well as any novel and newly identified strains that may be classified under that genus.

The term “biofilm” refers to an assemblage of microorganisms embedded in an extracellular polymer matrix and attached to a surface. A biofilm may comprise a single species of microbe or a plurality of species of microbes. The present disclosure provides for biofilms that form in a plant growth medium. This is taken to include biofilms that form independent of a plant part in the growth medium, and also biofilms that form in or on a part of a plant in the growth medium. Microbes that form biofilms include, but are not limited to, Achromobacter sp., Actinomyces sp., Agreia sp., Alcaligenes sp., Arthrobacter sp., Azomonas sp., Azorhizobium sp., Azospirillum sp., Bacillus sp., Brevibacterium sp., Burkholderia sp., Caballeronia sp., Cellulomonas sp., Cladosporium sp., Derxia sp., Desulfovibrio sp., Exiguobacterium sp., Flavobacterium sp., Leuconostoc sp., Micrococcus sp., Nitrosocosmicus sp., Paenibacillus sp., Paraburkholderia sp., Pseudomonas sp., Rhizobium sp., Rhodococcus sp., Rhodopseudomonas sp., Serratia sp., Sphingomonas sp., Xanthobacter sp.

The term “plant” is used in its broadest sense to refer to a terrestrial member of the kingdom Plantae, and includes bryophytes, pteridophytes and gymnosperms and angiosperms. A plant referred to herein may be a seed, a spore or a juvenile or adult plant originating from a seed or spore. A plant includes, but is not limited to, any species of grass, sedge, rush, ornamental or decorative, crop or cereal, fodder or forage, fruit or vegetable, fruit plant or vegetable plant, flower or vine or shrub or tree, exotic plant or house plant.

As used herein, the terms “crop”, “crop plant”, “cultivated plant” or “cultivated crop” are used in their broadest sense. The term includes, but is not limited to, any species of plant consumed by humans or used as a feed for land animals or fish or marine animals, or used by humans, or viewed by humans (e.g., flowers) or any plant used in industry or commerce or education, such as vegetable crop plants, fruit crop plants, fodder crop plants, fibre crop plants, and turf grass plants.

As used herein, the terms “plant growth medium” and “growth medium” are used interchangeably, and refer to any solid medium that supports the growth of a plant or a biofilm. The growth medium may be natural or artificial including, but not limited to, soil, potting mixes, bark, vermiculite and tissue culture gels. Naturally-occurring growth media may also include sand, mud, clay, humus, regolith and rock. An artificial growth medium may be constructed to mimic the conditions of a naturally occurring medium. Artificial growth media can be made from one or more of any number and combination of materials including sand, minerals, glass, rock, water, metals, salts, nutrients and water. The growth media may be used alone or in combination with one or more other media. It may also be used with or without the addition of exogenous nutrients and physical support systems for roots and foliage. In some embodiments the plant growth medium is soil or a nutrient extract from soil. The plant growth medium may be sterile or it may include a plurality of native microbes.

As used herein the term “rhizosphere” is used to denote that segment of the soil that surrounds the roots of a plant and is influenced by them. A rhizosphere may include compounds released by plant roots and also the microorganisms present in the soil environment and in and on the roots of a plant.

A “root volatile organic compound (rVOC)” referred to herein is a molecule with a low molecular weight and a high vapour pressure that is released by plant roots into the rhizosphere. An rVOC has a molecular weight in the range of about 100 to about 500 Da and a vapour pressure greater than 10 Pa at 293.15 K. It may be present as a gas or dissolved in a liquid within the rhizosphere. An rVOC may be produced and released by other parts of a plant in addition to the roots, and may be collected from the roots or another plant part for the purposes of the methods herein. Roots produce a number of VOCs, non-limiting examples of which include terpenoids, benzenoids, phenylpropanoids, glucosinolates, and oxylipins. Without being bound by theory, RVOCs may have a biological effect on the microbiome and/or on biofilms in a growth medium, e.g., it may promote or inhibit the growth of certain microbes, promote or inhibit the formation of biofilms, or change the microbial composition of a soil microbiome or soil biofilm. These changes may in turn affect the growth of a plant in the growth medium.

In one embodiment, there is provided a method for promoting plant growth in soil, the method comprising inducing biofilm growth by providing a root volatile organic compound (such as an oxylipin) to the soil.

As used herein, the term “plant growth promotion” encompasses a wide range of improved plant properties, including, but not limited to, improved nitrogen fixation, improved root development, increased leaf area, increased plant yield, increased seed germination, increased photosynthesis, improved resistance to plant pathogens, increase in accumulated biomass of the plant, or a combination thereof. The plant pathogen may include one or a combination of insects, nematodes, plant pathogenic fungi, or plant pathogenic bacteria. For a cultivated plant, plant yield refers to the amount of harvestable plant material or plant-derived product, and is normally defined as the measurable produce of economic value of the cultivated plant. For crop plants, yield also means the amount of harvested material per acre or unit of production. Yield may be defined in terms of quantity or quality. The harvested material may vary from crop to crop, for example, it may be seeds, aboveground biomass, roots, fruits, plant fibres, any other part of the plant, or any plant-derived product which is of economic value. The term “yield” also encompasses yield potential, which is the maximum obtainable yield. Yield may be dependent on a number of yield components, which may be monitored by certain parameters. These parameters are well known to persons skilled in the art and vary from crop to crop. The term “yield” also encompasses harvest index, which is the ratio between the harvested biomass over the total amount of biomass. In some embodiments, the method described herein leads to an increase in leaf and root area in a plant compared to a control plant that was not provided with an oxylipin.

As used herein, the term “biofilm growth” refers to an increase in the biomass or biovolume of a biofilm, which may derive from, but is not limited to, the growth of one or more microbial strains already present in a biofilm, the incorporation of additional microbial strains into a biofilm, the incorporation of previously planktonic microbes into a biofilm, the production of more extracellular material within a biofilm, or a combination thereof. In some embodiments, the application of an oxylipin leads to a biofilm growth of at least 5%, at least 10%, at least 25%, at least 50%, at least 75%, or at least 100%, as determined by an increase in biomass and/or biovolume of the biofilm.

In some embodiments, an rVOC used in the methods herein is selected from the group consisting of terpenoids, benzenoids, phenylpropanoids, glucosinolates, and oxylipins. In some embodiments an oxylipin is used in the methods herein.

An “oxylipin” of the present disclosure is a biologically active, oxygenated derivative of a polyunsaturated fatty acid, formed by oxidative metabolism of that fatty acid. Oxylipins are ubiquitous in animals, plants, algae, fungi and bacteria. In plants oxylipins may serve as signalling molecules regulating developmental processes like pollen formation or mediating responses to biotic and abiotic stresses.

In some embodiments, the oxylipin is a jasmonate or a derivative thereof. In some embodiments, the jasmonate is jasmonic acid, methyl jasmonate or dihydromethyl jasmonate, or a derivative thereof. In some embodiments the jasmonate is jasmonic acid, methyl jasmonate, dihydromethyl jasmonate, dihydro jasmonic acid, cis-jasmone, or a derivative thereof.

As used herein a “jasmonate” is a member of a family of oxylipins which is derived from or related to jasmonic acid. A jasmonate may be a natural or synthetic compound. Jasmonates include but are not limited to: jasmonic acid, methyl jasmonate (MeJA), dihydromethyl jasmonate (DHMJ), dihydro jasmonic acid, cis-jasmone, 7-iso-jasmonic acid, 9,10-dihydrojasmonic acid, 2,3-didehydrojasmonic acid, 3,4-didehydrojasmonic acid, 3,7-didehydrojasmonic acid, 4,5-didehydrojasmonic acid, 4,5-didehydro-7-isojasmonic acid, cucurbic acid, 6-epi-cucurbic acid lactones, 12-hydroxyjasmonic acid, 12-hydroxyjasmonic acid lactones, 11-hydroxyjasmonic acid, 8-hydroxyjasmonic acid, homo-jasmonic acid, dihomo-jasmonic acid, 11-hydroxy-dihomojasmonic acid, 8-hydroxy-dihomojasmonic acid, tuberonic acid, tuberonic acid-O-β-glucopyranoside, 5,6-didehydrojasmonic acid, 6,7-didehydrojasmonic acid, 7,8-didehydrojasmonic acid, dihydrojasmone, methylhydrojasmone, and jasmonic acid conjugated with amino acids. The compounds mentioned herein may contain a non-aromatic double bond and one or more asymmetric centres. Thus, they can occur as racemates and racemic mixtures, single enantiomers, individual diastereomers, diastereomeric mixtures, and cis- or trans-isomeric forms. All such isomeric forms are contemplated. For example, the jasmonate compound described herein includes all of any optical isomer that is based on the asymmetric carbon and is optically pure, any mixture of various optical isomers, or racemic form.

Methyl jasmonate (MeJA) and dihydromethyl jasmonate (DHMJ) are volatile jasmonates with known roles in plant communication in the aboveground parts of plants. MeJA has been applied exogenously to foliage to induce plant defensive responses and to maintain the post-harvest quality of fruits and vegetables. Disclosed herein is a role of MeJA and DHMJ in the belowground parts involving their effect on promoting biofilm growth in a plant growth medium.

The oxylipin may be provided to the growth medium as a solid, liquid or a gas. In many embodiments, the oxylipin will be applied in the form of an aqueous solution, but solid preparations, liquid suspensions, and preparations that allow the oxylipin to volatilise and expose the plant to oxylipin vapours may also be used. The oxylipin may be delivered in the form of emulsions, suspensions, solutions, powders, granules, pastes, aerosols and volatile formulations.

The oxylipin may be applied alone or in a formulation comprising other compounds. Some examples of other compounds that may be included in the formulation include wetting agents, adjuvants, emulsifiers, dispersants, spreaders, pastes, anchorage agents, coating agents, buffering agents, plant nutrients, and absorptive additives. The formulation may also include acids, bases, or other compounds that adjust or maintain the final pH of the formulation in order to increase solubility of certain compounds in the formulation or for other reasons. Those of skill in the art will recognise that a single ingredient may perform multiple functions, and may thus be classified or grouped in different ways. Particular examples of formulation ingredients include ionic, non-ionic, and zwitterionic surfactants, such as Triton® X-100, Triton® X-114, NP-40, Tween 20 (polysorbates) and sodium dodecyl sulfate; alcohols; and synthetic or natural oils, such as castor oil, canola (rapeseed) oil, and soybean oil. Citric acid may be used to acidify a formulation, and compounds such as dipotassium phosphate, calcium carbonate, and potassium silicate may be used to raise the pH.

The oxylipin preparation may be deposited or pumped into the growth medium or sprayed or fumigated or otherwise physically spread over the growth medium, by manual or mechanical means. The oxylipin preparation may be added in the vicinity of a plant or throughout the area of growth of a plant. The oxylipin may be applied once or repeatedly, depending on the formulation, the environmental conditions during and immediately after application, and the desired effect on biofilm and/or plant growth. A more dilute formulation may be used if repeated applications are to be performed.

In most embodiments the oxylipin is provided in an “effective amount” to promote plant growth. For the purposes of this disclosure, an effective amount of oxylipin is any amount of oxylipin that produces a quantifiable improvement in plant growth and/or a quantifiable increase in biofilm growth, compared to a control plant or a control biofilm which has not been provided with the oxylipin. It is understood by a skilled person that this effective concentration will not also induce any type of toxicity to the plant. In some embodiments, application of an effective amount of oxylipin leads to plant growth improvement that is an at least 5% increase, at least 10% increase, at least 25% increase, at least 50% increase, at least 75% increase, or at least a 100% increase in the property being measured. Thus, as non-limiting examples, the method according to this disclosure may produce an above-stated percentage increase in nitrogen fixation, or an above-stated increase in total root weight, or in leaf area or in plant product yield (e.g., an above-stated percentage increase in plant product weight), or an increased percentage of seeds that germinate, or rate of photosynthesis (e.g., determined by CO2 consumption) or accumulated biomass of the plant (e.g., determined by weight and/or height of the plant). The plant product is the item—usually but not necessarily—a food item produced by the plant.

In some embodiments, application of an effective amount an oxylipin leads to a biofilm growth of at least 5%, at least 10%, at least 25%, at least 50%, at least 75%, or at least 100%, as determined by an increase in biomass and/or biovolume of the biofilm.

The effective amount of oxylipin will vary depending on plant and microbial species and plant cultivar, and will depend on the manner of application, the form in which the oxylipin is administered, and the environmental conditions around the growth medium and/or around the plant that may include, for instance, the microbiome in the growth medium. Thus, different concentrations and exposure times for any given formulation will vary according to the type of plant and variety, and the type of microbes present in the growth medium. The concentration will also vary depending on the growth stage of the plant.

The growth medium, plant, microorganism and oxylipin may be combined or exposed to one another in any appropriate order. In one embodiment, the plant, seed, seedling, cutting, propagule or the like is planted or sown into a growth medium which already contains one or more microbe or which has previously been inoculated with one or more microbe, and the oxylipin is applied to the growth medium containing plant and microbe. In another embodiment, a microbe is inoculated into the growth medium and the oxylipin is applied to the growth medium to allow biofilm formation before the plant, seed, seedling, cutting, propagule or the like is planted or sown into the growth medium. In yet another embodiment, the plant, seed, seedling, cutting, propagule or the like is first planted or sown into the growth medium, allowed to grow, and at a later time one or more microbes are applied to the growth medium followed by the application of the oxylipin.

In some embodiments, the oxylipin is provided as a liquid. The oxylipin may be provided as an undiluted liquid or in the form of a solution with any compatible solvent, including aqueous (water) solutions, alcohol (e.g., ethanol) solutions, or in combinations of solvents (e.g., water/ethanol). A “compatible solvent” refers to any solvent in which the oxylipin is at least slightly soluble and which is not phytotoxic in the amounts or concentrations used for oxylipin application.

In one embodiment the oxylipin is provided in a single application to the growth medium. The amount of oxylipin added may vary depending on the extent of biofilm formation and/or plant growth desired.

In some embodiments the oxylipin is provided as a diluted liquid at a concentration of about 1 nM to about 10 μM. In some embodiments, the oxylipin is added as a diluted liquid at a concentration of about 1 nM to about 25 nM, about 1 nM to about 20 nM, about 1 nM to about 15 nM, about 1 nM to about 10 nM, or about 1 nM to about 5 nM. In some embodiments, the oxylipin is added as a diluted liquid at a concentration of about 0.5 μM to about 10 UM, about 0.6 UM to about 7 UM, 0.7 UM to about 5 UM, 0.8 UM to about 3 UM, about 0.9 UM to about 2 μM, about 0.95 UM to about 1.5 μM, or about 1 μM.

In some embodiments the oxylipin is provided as an undiluted liquid at a concentration of about 5 μmol to about 50 μmol, about 5 μmol to about 45 μmol, about 5 μmol to about 40 μmol, about 5 μmol to about 35 μmol, about 5 μmol to about 30 μmol, about 5 μmol to about 25 μmol, about 5 μmol to about 20 μmol, about 5 μmol to about 15 μmol, about 5 μmol to about 10 μmol, about 5 μmol to about 9 μmol, about 5 μmol to about 8 μmol, about 5 μmol to about 7 μmol, about 5 μmol to about 6 μmol, or about 5 μmol.

In some embodiments, the method further comprises a step of enriching the soil with plant growth microbes.

In some embodiments the plant is a species selected from bryophyte, pteridophyte, gymnosperm, monocot, and dicot. In some embodiments, the plant is a cultivated monocot or dicot.

In some embodiments, the dicot is, by non-limiting example, one of the following: bean, pea, tomato, pepper, squash, alfalfa, almond, anise seed, apple, apricot, arracha, artichoke, avocado, bambara groundnut, beet, bergamot, black pepper, black wattle, blackberry, blueberry, bitter orange, bok-choi, Brazil nut, breadfruit, broccoli, broad bean, Brussels sprouts, buckwheat, cabbage, camelina, Chinese cabbage, cacao, cantaloupe, caraway seeds, cardoon, carob, carrot, cashew nuts, cassava, castor bean, cauliflower, celeriac, celery, cherry, chestnut, chickpea, chicory, chili pepper, chrysanthemum, cinnamon, citron, clementine, clove, clover, coffee, cola nut, colza, corn, cotton, cottonseed, cowpea, crambe, cranberry, cress, cucumber, currant, custard apple, drumstick tree, earth pea, eggplant, endive, fennel, fenugreek, fig, filbert, flax, geranium, gooseberry, gourd, grape, grapefruit, guava, hemp, hempseed, henna, hop, horse bean, horseradish, indigo, jasmine, Jerusalem artichoke, jute, kale, kapok, kenaf, kohlrabi, kumquat, lavender, lemon, lentil, lespedeza, lettuce, lime, liquorice, litchi, loquat, lupine, macadamia nut, mace, mandarin, mangel, mango, medlar, melon, mint, mulberry, mustard, nectarine, niger seed, nutmeg, okra, olive, opium, orange, papaya, parsnip, pea, peach, peanut, pear, pecan nut, persimmon, pigeon pea, pistachio nut, plantain, plum, pomegranate, pomelo, poppy seed, potato, sweet potato, prune, pumpkin, quebracho, quince, trees of the genus Cinchona, quinoa, radish, ramie, rapeseed, raspberry, rhea, rhubarb, rose, rubber, rutabaga, safflower, sainfoin, salsify, sapodilla, Satsuma, scorzonera, sesame, shea tree, soybean, spinach, squash, strawberry, sugar beet, sugarcane, sunflower, swede, sweet pepper, tangerine, tea, teff, tobacco, tomato, trefoil, tung tree, turnip, urena, vetch, walnut, watermelon, yerba mate, wintercress, shepherd's purse, garden cress, peppercress, watercress, pennycress, star anise, laurel, bay laurel, cassia, jamun, dill, tamarind, peppermint, oregano, rosemary, sage, soursop, pennywort, calophyllum, balsam pear, kukui nut, Tahitian chestnut, basil, huckleberry, hibiscus, passionfruit, star apple, sassafras, cactus, St. John's wort, loosestrife, hawthorn, cilantro, curry plant, kiwi, thyme, zucchini, ulluco, jicama, waterleaf, spiny monkey orange, yellow mombin, starfruit, amaranth, wasabi, Japanese pepper, yellow plum, mashua, Chinese toon, New Zealand spinach, bower spinach, ugu, tansy, chickweed, jocote, Malay apple, paracress, sowthistle, Chinese potato, horse parsley, hedge mustard, campion, agate, cassod tree, thistle, burnet, star gooseberry, saltwort, glasswort, sorrel, silver lace fern, collard greens, primrose, cowslip, purslane, knotgrass, terebinth, tree lettuce, wild betel, West African pepper, yerba santa, tarragon, parsley, chervil, land cress, burnet saxifrage, honeyherb, butterbur, shiso, water pepper, perilla, bitter bean, oca, kampong, Chinese celery, lemon basil, Thai basil, water mimosa, cicely, cabbage-tree, moringa, mauka, ostrich fern, rice paddy herb, yellow sawah lettuce, lovage, pepper grass, maca, bottle gourd, hyacinth bean, water spinach, catsear, fishwort, Okinawan spinach, lotus sweetjuice, gallant soldier, culantro, arugula, cardoon, caigua, mitsuba, chipilin, samphire, mampat, ebolo, ivy gourd, cabbage thistle, sea kale, chaya, huauzontle, Ethiopian mustard, magenta spreen, good king henry, epazole, lamb's quarters, centella plumed cockscomb, caper, rapini, napa cabbage, mizuna, Chinese savoy, kai-lan, mustard greens, Malabar spinach, chard, marshmallow, climbing wattle, China jute, paprika, annatto seed, spearmint, savory, marjoram, cumin, chamomile, lemon balm, allspice, bilberry, cherimoya, cloudberry, damson, pitaya, durian, elderberry, feijoa, jackfruit, jambul, jujube, physalis, purple mangosteen, rambutan, redcurrant, blackcurrant, salal berry, satsuma, ugli fruit, azuki bean, black bean, black-eyed pea, borlotti bean, common bean, green bean, kidney bean, lima bean, mung bean, navy bean, pinto bean, runner bean, mangetout, snap pea, broccoflower, calabrese, nettle, bell pepper, raddichio, daikon, white radish, skirret, tat soi, broccolini, black radish, burdock root, fava bean, broccoli raab, lablab, lupin, sterculia, velvet beans, winged beans, yam beans, mulga, ironweed, umbrella bush, tjuntjula, wakalpulka, witchetty bush, wiry wattle, chia, beech nut, candlenut, colocynth, mamoncillo, Maya nut, mongongo, ogbono nut, paradise nut, and cempedak.

In some embodiments, the dicot is from, by non-limiting example, one of the following families: Acanthaceae (acanthus), Aceraceae (maple), Achariaceae, Achatocarpaceae (achatocarpus), Actinidiaceae (Chinese gooseberry), Adoxaceae (moschatel), Aextoxicaceae, Aizoaceae (fig marigold), Akaniaceae, Alangiaceae, Alseuosmiaceae, Alzateaceae, Amaranthaceae (amaranth), Amborellaceae, Anacardiaceae (sumac), Ancistrocladaceae, Anisophylleaceae, Annonaceae (custard apple), Apiaceae (carrot), Apocynaceae (dogbane), Aquifoliaceae (holly), Araliaceae (ginseng), Aristolochiaceae (birthwort), Asclepiadaceae (milkweed), Asteraceae (aster), Austrobaileyaceae, Balanopaceae, Balanophoraceae (balanophora), Balsaminaceae (touch-me-not), Barbeyaceae, Barclayaceae, Basellaceae (basella), Bataceae (saltwort), Begoniaceae (begonia), Berberidaceae (barberry), Betulaceae (birch), Bignoniaceae (trumpet creeper), Bixaceae (lipstick tree), Bombacaceae (kapok tree), Boraginaceae (borage), Brassicaceae (mustard, also Cruciferae), Bretschneideraceae, Brunelliaceae (brunellia), Bruniaceae, Brunoniaceae, Buddlejaceae (butterfly bush), Burseraceae (frankincense), Buxaceae (boxwood), Byblidaceae, Cabombaceae (water shield), Cactaceae (cactus), Caesalpiniaceae, Callitrichaceae (water starwort), Calycanthaceae (strawberry shrub), Calyceraceae (calycera), Campanulaceae (bellflower), Canellaceae (canella), Cannabaceae (hemp), Capparaceae (caper), Caprifoliaceae (honeysuckle), Cardiopteridaceae, Caricaceae (papaya), Caryocaraceae (souari), Caryophyllaceae (pink), Casuarinaceae (she-oak), Cecropiaceae (cecropia), Celastraceae (bittersweet), Cephalotaceae, Ceratophyllaceae (hornwort), Cercidiphyllaceae (katsura tree), Chenopodiaceae (goosefoot), Chloranthaceae (chloranthus), Chrysobalanaceae (cocoa plum), Circaeasteraceae, Cistaceae (rockrose), Clethraceae (clethra), Clusiaceae (mangosteen, also Guttiferae), Cneoraceae, Columelliaceae, Combretaceae (Indian almond), Compositae (aster), Connaraceae (cannarus), Convolvulaceae (morning glory), Coriariaceae, Cornaceae (dogwood), Corynocarpaceae (karaka), Crassulaceae (stonecrop), Crossosomataceae (crossosoma), Crypteroniaceae, Cucurbitaceae (cucumber), Cunoniaceae (cunonia), Cuscutaceae (dodder), Cyrillaceae (cyrilla), Daphniphyllaceae, Datiscaceae (datisca), Davidsoniaceae, Degeneriaceae, Dialypetalanthaceae, Diapensiaceae (diapensia), Dichapetalaceae, Didiereaceae, Didymelaceae, Dilleniaceae (dillenia), Dioncophyllaceae, Dipentodontaceae, Dipsacaceae (teasel), Dipterocarpaceae (meranti), Donatiaceae, Droseraceae (sundew), Duckeodendraceae, Ebenaceae (ebony), Elaeagnaceae (oleaster), Elaeocarpaceae (elaeocarpus), Elatinaceae (waterwort), Empetraceae (crowberry), Epacridaceae (epacris), Eremolepidaceae (catkin-mistletoe), Ericaceae (heath), Erythroxylaceae (coca), Eucommiaceae, Eucryphiaceae, Euphorbiaceae (spurge), Eupomatiaceae, Eupteleaceae, Fabaceae (pea or legume), Fagaceae (beech), Flacourtiaceae (flacourtia), Fouquieriaceae (ocotillo), Frankeniaceae (frankenia), Fumariaceae (fumitory), Garryaceae (silk tassel), Geissolomataceae, Gentianaceae (gentian), Geraniaceae (geranium), Gesneriaceae (gesneriad), Globulariaceae, Gomortegaceae, Goodeniaceae (goodenia), Greyiaceae, Grossulariaceae (currant), Grubbiaceae, Gunneraceae (gunnera), Gyrostemonaceae, Haloragaceae (water milfoil), Hamamelidaceae (witch hazel), Hernandiaceae (hernandia), Himantandraceae, Hippocastanaceae (horse chestnut), Hippocrateaceae (hippocratea), Hippuridaceae (mare's tail), Hoplestigmataceae, Huaceae, Hugoniaceae, Humiriaceae, Hydnoraceae, Hydrangeaceae (hydrangea), Hydrophyllaceae (waterleaf), Hydrostachyaceae, Icacinaceae (icacina), Idiospermaceae, Illiciaceae (star anise), Ixonanthaceae, Juglandaceae (walnut), Julianiaceae, Krameriaceae (krameria), Lacistemataceae, Lamiaceae (mint, also Labiatae), Lardizabalaceae (lardizabala), Lauraceae (laurel), Lecythidaceae (brazil nut), Leeaceae, Leitneriaceae (corkwood), Lennoaceae (lennoa), Lentibulariaceae (bladderwort), Limnanthaceae (meadow foam), Linaceae (flax), Lissocarpaceae, Loasaceae (loasa), Loganiaceae (logania), Loranthaceae (showy mistletoe), Lythraceae (loosestrife), Magnoliaceae (magnolia), Malesherbiaceae, Malpighiaceae (barbados cherry), Malvaceae (mallow), Marcgraviaceae (shingle plant), Medusagynaceae, Medusandraceae, Melastomataceae (melastome), Meliaceae (mahogany), Melianthaceae, Mendonciaceae, Menispermaceae (moonseed), Menyanthaceae (buckbean), Mimosaceae, Misodendraceae, Mitrastemonaceae, Molluginaceae (carpetweed), Monimiaceae (monimia), Monotropaceae (Indian pipe), Moraceae (mulberry), Moringaceae (horseradish tree), Myoporaceae (myoporum), Myricaceae (bayberry), Myristicaceae (nutmeg), Myrothamnaceae, Myrsinaceae (myrsine), Myrtaceae (myrtle), Nelumbonaceae (lotus lily), Nepenthaceae (East Indian pitcherplant), Neuradaceae, Nolanaceae, Nothofagaceae, Nyctaginaceae (four-o'clock), Nymphaeaceae (water lily), Nyssaceae (sour gum), Ochnaceae (ochna), Olacaceae (olax), Oleaceae (olive), Oliniaceae, Onagraceae (evening primrose), Oncothecaceae, Opiliaceae, Orobanchaceae (broom rape), Oxalidaceae (wood sorrel), Paeoniaceae (peony), Pandaceae, Papaveraceae (poppy), Papilionaceae, Paracryphiaceae, Passifloraceae (passionflower), Pedaliaceae (sesame), Pellicieraceae, Penaeaceae, Pentaphragmataceae, Pentaphylacaceae, Peridiscaceae, Physenaceae, Phytolaccaceae (pokeweed), Piperaceae (pepper), Pittosporaceae (pittosporum), Plantaginaceae (plantain), Platanaceae (plane tree), Plumbaginaceae (leadwort), Podostemaceae (river weed), Polemoniaceae (phlox), Polygalaceae (milkwort), Polygonaceae (buckwheat), Portulacaceae (purslane), Primulaceae (primrose), Proteaceae (protea), Punicaceae (pomegranate), Pyrolaceae (shinleaf), Quiinaceae, Rafflesiaceae (rafflesia), Ranunculaceae (buttercup orranunculus), Resedaceae (mignonette), Retziaceae, Rhabdodendraceae, Rhamnaceae (buckthorn), Rhizophoraceae (red mangrove), Rhoipteleaceae, Rhynchocalycaceae, Rosaceae (rose), Rubiaceae (madder), Rutaceae (rue), Sabiaceae (sabia), Saccifoliaceae, Salicaceae (willow), Salvadoraceae, Santalaceae (sandalwood), Sapindaceae (soapberry), Sapotaceae (sapodilla), Sarcolaenaceae, Sargentodoxaceae, Sarraceniaceae (pitcher plant), Saururaceae (lizard's tail), Saxifragaceae (saxifrage), Schisandraceae (schisandra), Scrophulariaceae (figwort), Scyphostegiaceae, Simaroubaceae (quassia), Simmondsiaceae (jojoba), Scytopetalaceae, Solanaceae (potato), Sonneratiaceae (sonneratia), Sphaerosepalaceae, Sphenocleaceae (spenoclea), Stackhousiaceae (stackhousia), Stachyuraceae, Staphyleaceae (bladdernut), Sterculiaceae (cacao), Stylidiaceae, Styracaceae (storax), Surianaceae (suriana), Symplocaceae (sweetleaf), Tamaricaceae (tamarix), Tepuianthaceae, Tetracentraceae, Tetrameristaceae, Theaceae (tea), Theligonaceae, Theophrastaceae (theophrasta), Thymelaeaceae (mezereum), Ticodendraceae, Tiliaceae (linden), Tovariaceae, Trapaceae (water chestnut), Tremandraceae, Trigoniaceae, Trimeniaceae, Trochodendraceae, Tropaeolaceae (nasturtium), Turneraceae (turnera), Ulmaceae (elm), Urticaceae (nettle), Valerianaceae (valerian), Verbenaceae (verbena), Violaceae (violet), Viscaceae (Christmas mistletoe), Vitaceae (grape), Vochysiaceae, Winteraceae (wintera), Xanthophyllaceae, and Zygophyllaceae (creosote bush).

In some embodiments, the monocot is, by non-limiting example, one of the following: corn, wheat, oat, rice, barley, millet, banana, onion, garlic, asparagus, ryegrass, millet, fonio, raishan, nipa grass, turmeric, saffron, galangal, chive, cardamom, date palm, pineapple, shallot, leek, scallion, water chestnut, ramp, Job's tears, bamboo, ragi, spotless watermeal, arrowleaf elephant ear, Tahitian spinach, abaca, areca, bajra, betel nut, broom millet, broom sorghum, citronella, coconut, cocoyam, maize, dasheen, durra, durum wheat, edo, Pique, formio, ginger, orchard grass, esparto grass, Sudan grass, guinea corn, Manila hemp, henequen, hybrid maize, jowar, lemon grass, maguey, bulrush millet, finger millet, foxtail millet, Japanese millet, proso millet, New Zealand flax, oats, oil palm, palm palmyra, sago palm, redtop, sisal, sorghum, spelt wheat, sweet corn, sweet sorghum, taro, teff, timothy grass, triticale, vanilla, wheat, and yam.

In some embodiments, the monocot is from, by non-limiting example, one of the following families: Acoraceae (calamus), Agavaceae (century plant), Alismataceae (water plantain), Aloeaceae (aloe), Aponogetonaceae (cape pondweed), Araceae (arum), Arecaceae (palm), Bromeliaceae (bromeliad), Burmanniaceae (burmannia), Butomaceae (flowering rush), Cannaceae (canna), Centrolepidaceae, Commelinaceae (spiderwort), Corsiaceae, Costaceae (costus), Cyanastraceae, Cyclanthaceae (Panama hat), Cymodoceaceae (manatee grass), Cyperaceae (sedge), Dioscoreaceae (yam), Eriocaulaceae (pipewort), Flagellariaceae, Geosiridaceae, Haemodoraceae (bloodwort), Hanguanaceae (hanguana), Heliconiaceae (heliconia), Hydatellaceae, Hydrocharitaceae (tape grass), Iridaceae (iris), Joinvilleaceae (joinvillea), Juncaceae (rush), Juncaginaceae (arrow grass), Lemnaceae (duckweed), Liliaceae (lily), Limnocharitaceae (water poppy), Lowiaceae, Marantaceae (prayer plant), Mayacaceae (mayaca), Musaceae (banana), Najadaceae (water nymph), Orchidaceae (orchid), Pandanaceae (screw pine), Petrosaviaceae, Philydraceae (philydraceae), Poaceae (grass), Pontederiaceae (water hyacinth), Posidoniaceae (posidonia), Potamogetonaceae (pondweed), Rapateaceae, Restionaceae, Ruppiaceae (ditch grass), Scheuchzeriaceae (scheuchzeria), Smilacaceae (catbrier), Sparganiaceae (bur reed), Stemonaceae (stemona), Strelitziaceae, Taccaceae (tacca), Thurniaceae, Triuridaceae, Typhaceae (cattail), Velloziaceae, Xanthorrhoeaceae, Xyridaceae (yellow-eyed grass), Zannichelliaceae (horned pondweed), Zingiberaceae (ginger), and Zosteraceae (eelgrass).

Disclosed herein is also a method for inducing biofilm growth in a growth medium, the method comprising providing an oxylipin to the growth medium, wherein the oxylipin induces biofilm growth in the growth medium.

In some embodiments, the biofilm growth that is induced by an oxylipin is capable of promoting plant growth in the growth medium. The biofilm may have a plant-beneficial effect without growing on any parts of a plant. The biofilm may, for instance, grow in the vicinity of plant roots and produce growth-promoting VOCs that are absorbed by plant parts underground and/or aboveground.

In some embodiments the plant is introduced to the growth medium after the growth of the biofilm. In some embodiments, the plant may induce changes to biofilm composition, which may include, but is not limited to, changes to the number and diversity of microbes in the biofilm, the behaviour and/or metabolic activity of microbes in the biofilm, the amount of extracellular material in the biofilm, the biomass or biovolume of the biofilm. These plant-induced biofilm changes may result in beneficial effects on the plant.

In some embodiments, the biofilm is capable of sequestering one or more plant pathogens. Pathogenic microbes may include, but are not limited to, plant fungal pathogens, plant bacterial pathogens, Alternaria sp., Aspergillus sp., Botrytis sp., Cercospora sp., Claviceps sp., Erwinia sp., Fusarium sp., Glomerella sp., Macrophomina sp., Magnaorthe sp., Pantoea sp., Phoma sp., Phytophthora sp., Pythium sp., Ralstonia sp., Rhizoctonia sp., Tilletia sp., Ustilago sp., Xanthomonas sp. The biofilm may sequester plant pathogens preferentially over other microbes, including plant-beneficial microbes.

Disclosed herein is a growth medium for promoting plant growth, wherein the growth medium comprises an oxylipin.

Disclosed herein is a kit for promoting plant growth, wherein the kit comprises a growth medium, wherein the growth medium comprises an oxylipin.

Disclosed herein is a method for isolating a microbe that is responsive to a root volatile organic compound, the method comprising culturing a plurality of microbes in a growth medium comprising a root volatile organic compound, isolating a microbe that is enriched in the growth medium as compared to a growth medium that does not contain the root volatile organic compound.

In one embodiment, the method comprises identifying the isolated microbe. The isolated microbe may be identified by sequencing. This may be done by sequencing the 16S rRNA gene segments from genomic DNA isolated from the microbe.

In some embodiments, the isolated microbe is capable of promoting plant growth in a growth medium.

It will be appreciated that many further modifications and permutations of various aspects of the described embodiments are possible. Accordingly, the described aspects are intended to embrace all such alterations, modifications, and variations that fall within the spirit and scope of the appended claims.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary elements of the invention. The phrase allows for the presence of other non-recited elements which do not materially affect the characteristics of the invention but excludes additional unspecified elements which would affect the basic and novel characteristics of the method defined.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Certain embodiments of the invention will now be described with reference to the following examples which are intended for the purpose of illustration only and are not intended to limit the scope of the generality hereinbefore described.

### EXAMPLES

### Methods

### Soil Extract Medium and Agar

Soil extract medium was prepared by autoclaving 70 g of JIFFY soil substrate in 1 liter of water. It was cooled down and was then filtered through a 0.22 μm Nalgene filtration unit. For preparing soil extract agar plates, 1% (w/v) agarose was added to the filtered media and the solution was autoclaved again before pouring into the plates. This is the default broth and agar medium for all the experiments in the manuscript unless stated otherwise.

### Soil Microbiota Inoculum Preparation

5 g of JIFFY soil substrate was resuspended in 20 ml of PBS. This suspension was vortexed for 4 minutes and sonicated for 1 minute. It was then filtered through a strainer. The slurry that didn't pass through the strainer was again resuspended in 20 ml of PBS medium and subsequent steps were repeated twice. The filtrate was then centrifuged at 150G for 2 minutes to settle large soil particles and the supernatant was decanted into another tube. This supernatant was then centrifuged at 5500G for 5 minutes to obtain a bacterial pellet. This pellet was resuspended in 20 ml of soil extract medium. This suspension was referred to as “soil microbiota inoculum”. The final concentration in all inocula was around 1×108 bacteria per ml as quantified by Baclight bacterial counting kit (flow cytometry) or manual counting with a hemocytometer.

For the confocal imaging experiments, soil inoculum was enriched in soil extract media overnight at 37° C.

### Plant Species and Mutants

Arabidopsis insertional mutant lines were acquired from Arabidopsis Biological Research Centre at Ohio State University (details mentioned in key resource table) and segregated for homozygous lines wherever viable. In most cases, Arabidopsis thaliana (col-0) and mutant seedlings were grown in pots with Jiffy universal potting soil up to 12 days in a plant growth chamber with the following settings: 16 hours of light at 23° C. followed by 8 hours of darkness at 21° C. with 80% relative humidity. Tomato, tobacco, rice, and fern were grown similarly. For in vitro experiments, plants were grown in soil extract agar (preparation described below). Before germination, plants were surface sterilised with 50% Chlorox and stratified for two days at 4° C.

### Static System Assembly

This system is a modification of the bipartite system (Ryu, C. M. et al., Proc. Natl. Acad. Sci. U.S.A 100, 4927-4932 (2003)) that is routinely used to study microbial VOCs. Circular Petri plates (90 mm diameter) were filled with MS media, and Arabidopsis seeds were grown on it (post-sterilisation) for 12 days. A square portion of MS media was cut out and a smaller Petri plate (35 mm diameter) with microbial inoculum (1 ml) was placed in it. There was sufficient headspace to allow for gaseous exchange. The lid was then tightly closed with parafilm to avoid the loss of VOCs. At particular time points, the smaller plates were taken out, and biofilm was quantified with crystal violet staining assay (as described below).

### Dynamic System Assembly

This system is an implementation of designs proposed in previous reviews (Delory, B. M. et al., Plant Soil 402, 1-26 (2016); Tholl, D. et al., Plant J. 45, 540-560, (2006)).

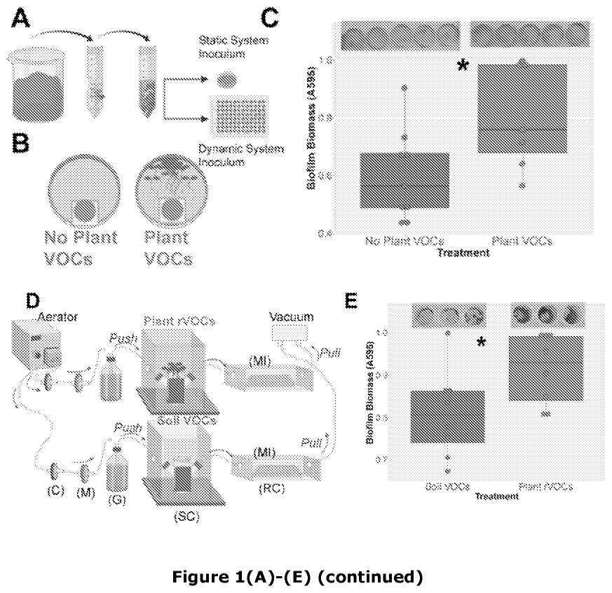

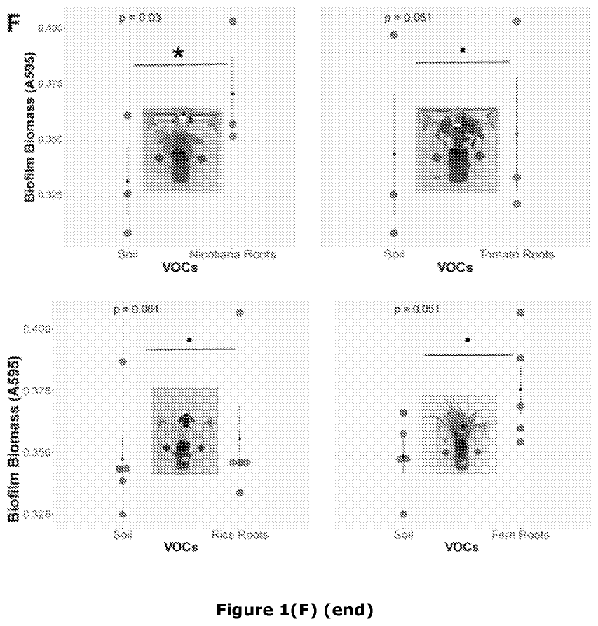

This system consists of an aerator/pump (to push air), 5 μm charcoal filter (to adsorb gaseous impurities), 0.22 μm filter (to trap microbial contamination), gas wash bottle (to moisturize the air), a source chamber (to host the source of volatiles), recipient chamber (to receive volatiles) and vacuum pump (to pull the air out) (FIG. 1D). All the modules can be interconnected through silicone tubes to create a unidirectional continuous flow of sterile air with the help of a pump (push) and vacuum (pull). A bunch of 100-150 2-week old seedlings with rhizosphere soil were kept in a customised glass pot having two open side arms which were used as inlet and outlet of the air with the roots in between. The microbiome suspension in the microtitre plate with 96 wells was kept within the recipient chamber. The whole glass pot with plants was put into the source chamber. The source chamber with only soil was taken as a control to study the effects of rVOCs on soil microbiome biofilm formation.

Charcoal filters (5 μm) and polytetrafluoroethylene (PTFE) filters (0.22 μm) were procured from Omega Scientific Pte Ltd, Singapore. The source chambers and receiving chambers were custom made by Million Fabricators, Singapore. Silicone tubings were used to connect all parts of the system. Airflow from the inlet (aerator) and outlet (vacuum) of the pot was measured using a mechanical flowmeter to be around 400 ml/min.

### Crystal Violet (CV) Staining for Biofilm Biomass Estimation

This method was used to get a proxy for biofilm biomass. Briefly, planktonic cells were discarded. 50 μL of 0.1% CV solution was added to the well very gently. The biofilm was stained for 10 minutes. The dye was removed gently. 100 μL of PBS was added to the well to wash off the excess CV. PBS was removed, and the wells were left to dry overnight. The next day, 200 ul of 1% SDS was added to each well and was resuspended vigorously with a pipette. After 20 minutes, 20 ul from the top suspension was removed and added to a new 96-well plate. The well was diluted with 180 ul of water and absorbance was checked at 595 nm on a spectrophotometer.

Volatile trapping and TD-GCMS

rVOCs and soil VOCs were trapped as described previously (Schulz-Bohm, K. et al., ISME J. 1-11 (2018)). Briefly, 2 Tenax cartridges were fitted into the side-arms of the glass pots in such a way that their opening is exposed towards the plant roots/soil. VOCs were sampled for 40 hours and immediately analysed by thermal desorption-gas chromatography-mass spectrometry.

Sample preparation and injection were performed using the fully automated Gerstel MPS-2 autosampler and Gerstel MAESTRO software. Volatile compounds were adsorbed on a Tenax TA tube. A thermal desorption unit (TDU) was used to thermally desorb the volatiles in splitless mode at 230° C. for 10 min. To ensure that the volatiles released from the TDU are quantitatively trapped, the cooled injection system-programmed temperature vaporiser (CIS-PTV) was used. The CIS was heated from 80° C. to 230° C. at the rate of 12° C./sec with the split valve closed during sample injection into the GC inlet. Analyses of volatile compounds were performed on an Agilent 7890B GC coupled to a 5977B quadruple mass spectrometer. Separation of compounds was performed on a DB-FFAP column (60 m×250 μm×0.25 μm, Agilent Technologies, Middleburg, OI, USA). Helium was used as the carrier gas at a flow rate of 1.9 ml/min and the solvent vent mode was used. The inlet temperature was 250° C. The oven program was as follows: initial temperature 50° C. held for 1 minute, then increased to 230° C. at the rate of 10° C./min and held for 20 minutes. The temperature of the ion source and transfer line was 250° C.

The mass spectrometer was in electron ionisation mode with an ionization energy of 70 eV, scan range of 40-300 m/z and solvent delay of 3.75 minutes. Analysis was performed in Single Ion Monitoring (SIM) mode by monitoring the following ions 83, 151.1, 224.1 with the dwell time of 150 ms. Mass Hunter Qualitative Analysis was used to extract and integrate peak spectra. Peak area of these ions was considered for the relative quantification of MeJA among different samples.

### Live Imaging of Biofilm and Matrix Formation

Soil microbiota inoculum was prepared as described in the section above. MeJA was added to the microbiota to achieve the desired concentration (0, 1, 5, 25 nM for nucleic acid imaging experiment, and 0 and 5 nM for matrix imaging experiment). 50 L of microbiota suspension was added to every well of Ibidi™ p-Slide 18 Well (Cat. No: 81816) that had a cover glass bottom. 50 UL of SYTO™9 (Thermo cat. no. S34854) solution (final concentration of 5 μM) was also added to all the wells. For matrix imaging, FilmTracer™ SYPRO™ Ruby Biofilm Matrix Stain (Thermo, cat. no. F10318) was added instead (ready to use, 1×concentration). For live imaging, Zeiss LSM 900 with Airyscan (Definite Focus 2) was used and images were acquired every 30 minutes for 24 hours with 65×oil objective at the NUS Centre for Bioimaging Science (CBIS). For both the dyes (separate experiments), a 488 nm laser was used.

### Biofilm Image Analysis

Image analysis was performed using BiofilmQ software (Hartmann, A. et al., Plant Soil 312, 7-14 (2008)). Images were aligned along the Z-axis and along time. 2-class Otsu thresholding was used to detect the signal against the background. Sensitivity was set based on thresholding feedback. The rest of the settings were kept at default. Biofilm-related global properties were calculated and exported. We mainly focused on the 3D biovolume of our samples. Linear mixed-effects were used to model the biovolume where time, treatment, and their interaction were the fixed effects, and every sample was considered a random effect (Table 1 and Table 2). Following packages from R were used: nlme (Pinheiro, J. et al., R package version 3.1-153 (2021)), ggplot2 (Wickham, H., Wiley Interdiscip. Rev. Comput. Stat. 3, 180-185 (2011)).

Identification of rVOCs- and MeJA-Responder Strains

Using the push-pull airflow system, soil microbiota inoculum was exposed to four VOC treatments 1) soil VOCs; 2) WT Arabidopis rVOCs; 3) jmt Arabidopsis rVOCs; 4) jmt Arabidopsis rVOCs+MeJA. Biofilm and planktonic parts of the samples were collected from 28 wells after 16 and 24 hours and stored at −80° C. The collective sample from 28 wells was treated as a single experimental replicate. The whole experiment was repeated eight times.

Biofilm DNA Extraction and 16S rRNA Gene Amplicon Sequencing

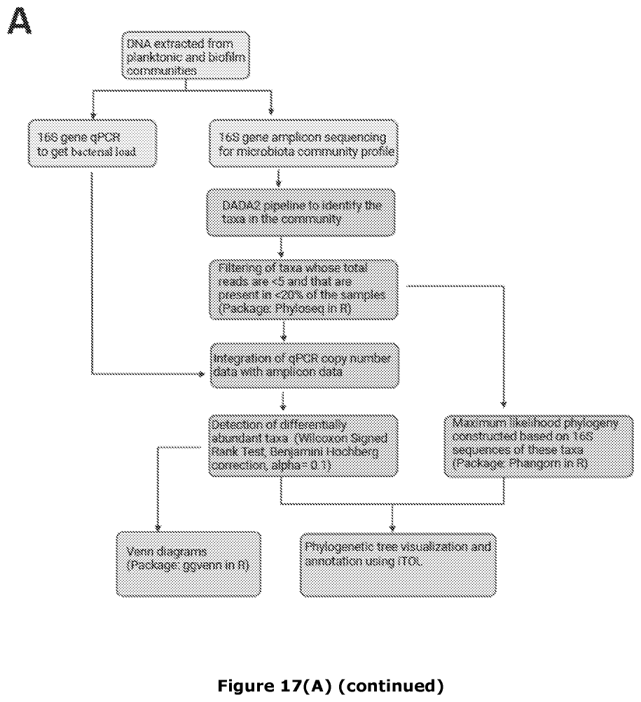

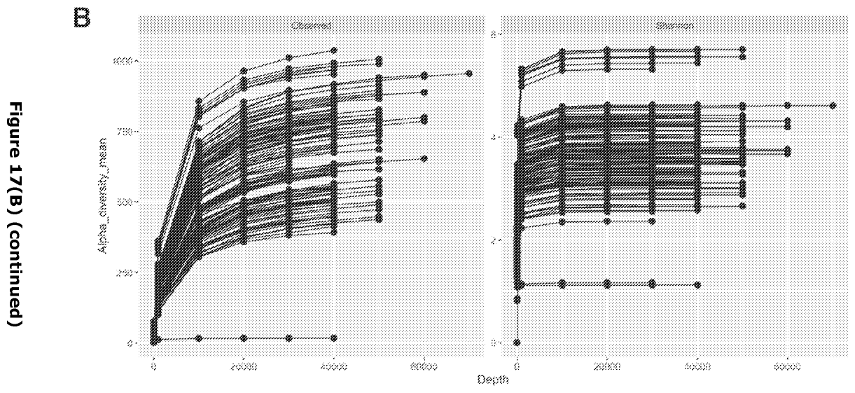

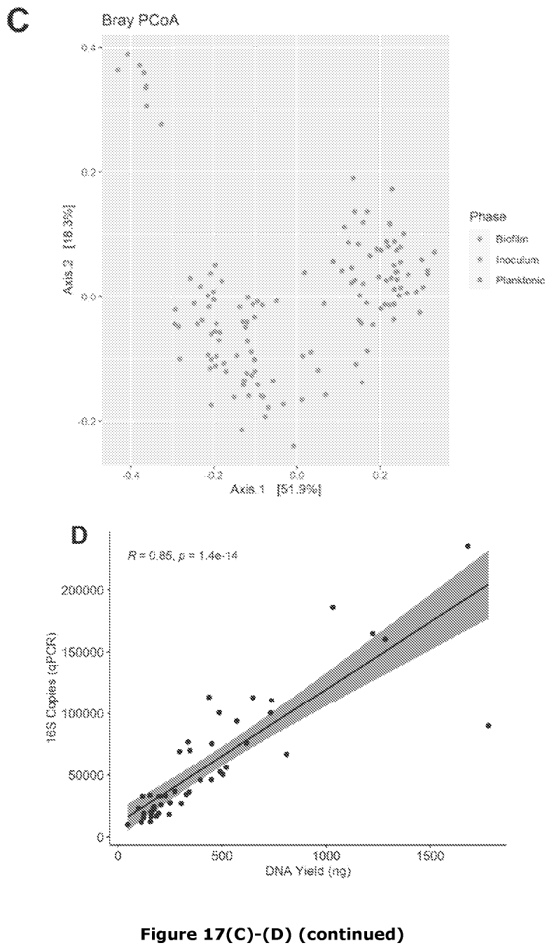

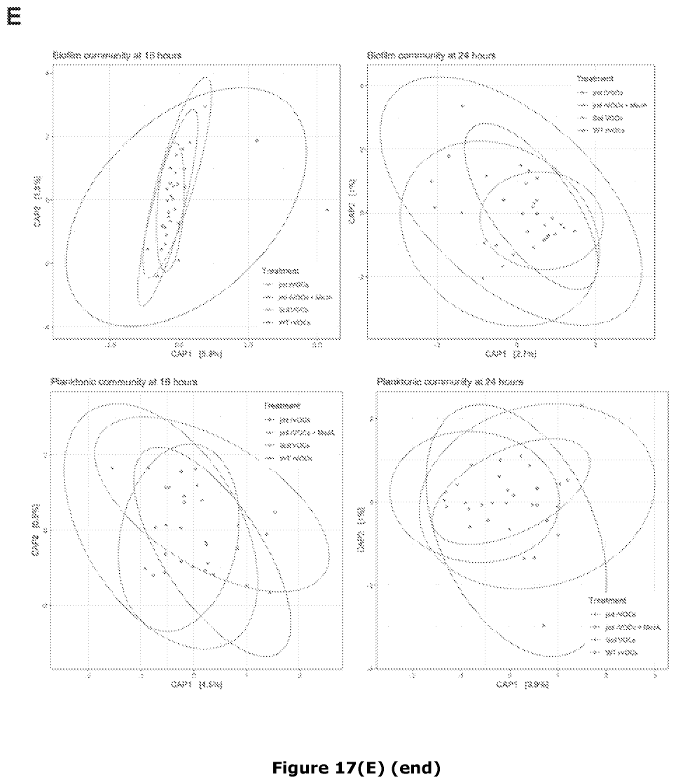

Biofilm was scraped at specific timepoints and resuspended in PBS solution. DNA-RNA shield was added in 1:1 proportion and samples were stored at −80 C. Zymobiomics DNA miniprep kit was used to isolate DNA from the samples based on their protocol. 16S V4-V5 region was amplified using 515F-Y and 927R primers (Walters et al., mSystems 1 (2016)). 20 UL of reaction contained 2 μL of 10×DreamTaq buffer, 2 μL of 2 mM dNTP mix, 0.5 μL of each primer (10 UM), 0.5 μL of DreamTaq polymerase (5U/μL), 10 ng of template DNA, and molecular grade water to make up the volume. PCR conditions were as follows: initial denaturation at 95° C. for 3 minutes, 35 cycles of denaturation at 95° C. for 45 s, annealing at 50° C. for 45 s, extension at 68° C. for 90 s and final extension at 68° C. for 5 minutes. PCR products were purified with a Genejet PCR purification kit. Amplicon concentration was measured using Qubit DNA BR Kit and Qubit fluorometer. 16S amplicons were submitted for next-gen sequencing on Illumina MiSeq V3 Run (300 base pairs paired-end) at Singapore Centre for Life Science Engineering (SCELSE). Rarefaction analysis was performed to calculate appropriate depth for sequencing (FIG. 17B).

qPCR for 16S Copies (Bacterial Load)

To enumerate the 16S rRNA gene copy numbers, the primer pair 515F and 806R (Walters, W. et al., MSystems 1, (2016)) were used in qPCR to amplify the 16S gene using Applied Biosystem real-time PCR system. The PCR assay mixture consists of 10 ul of PowerUp™ SYBR™ Green Master Mix, 1 ul of each primer from 10 UM stock, 1 ul of DNA of extracted DNA from the microbial population, and 7 ul of sterile nuclease-free water. The PCR amplification program encompassed an initial denaturation step at 95° C. for 3 min followed by 40 three-step cycles at 95° C. for 30 s, at 52° C. for 30 s and 72° C. for 30 s. Plasmid with the fragments of 16S rRNA gene part amplified with same primer pair was taken as standard for creating standard curve with known copy number for absolute quantification. Pearson correlation was calculated for the qPCR-derived copy number and the DNA yield from all the samples (FIG. 17D).

### Microbiome Sequencing Data Analysis

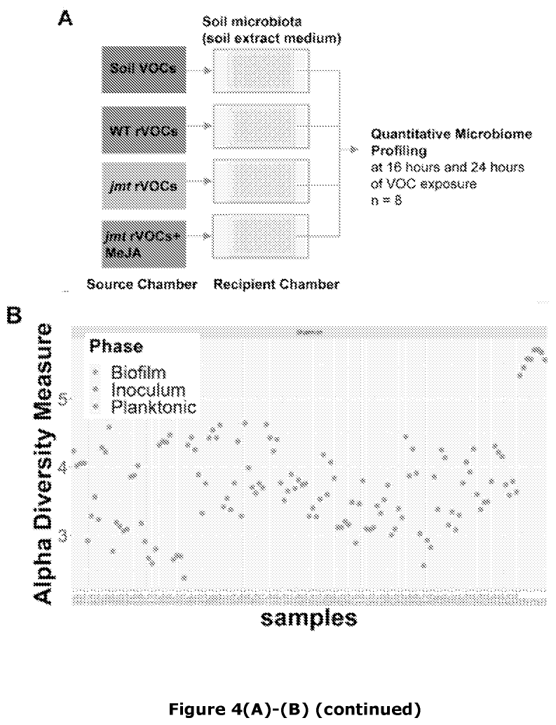

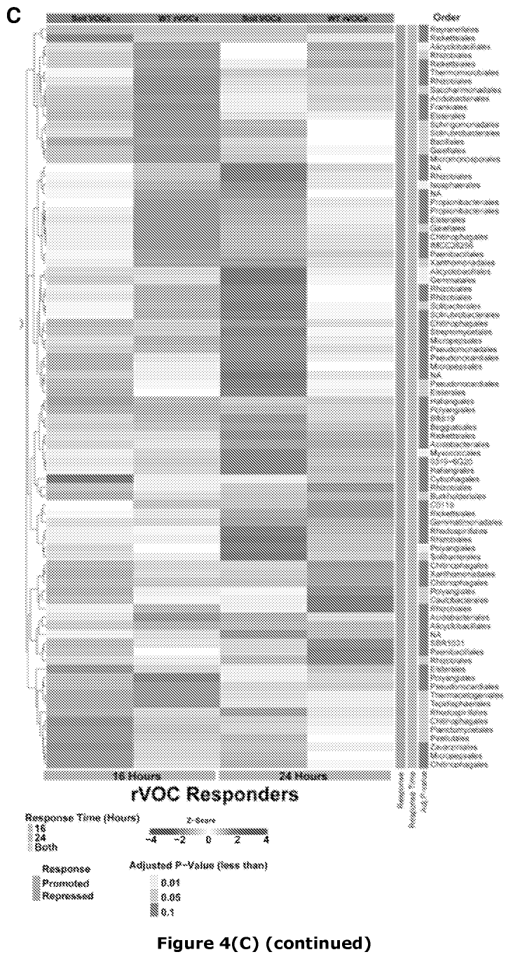

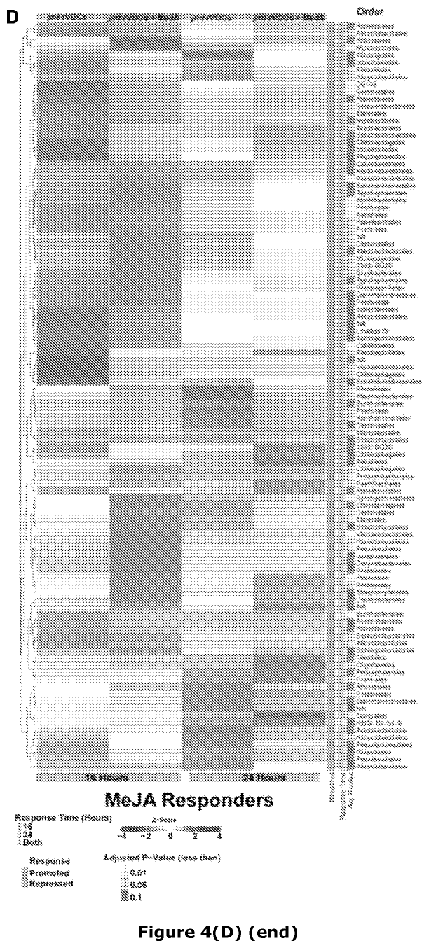

Raw and demultiplexed sequencing data was analysed as follows (also described in a flowchart in FIG. 17A): Primer and adapter sequences were removed using cutadapt (Martin, M., EMBnet.journal 17, 10-12 (2011)). DADA2 (Callahan, B. J. et al., Nat. Methods 13, 581-583 (2016)) pipeline was used to learn the error rates and get amplicon sequence variants (ASVs). Silva database was used to map the ASVs to their phylogeny. Thereafter, statistical analysis was performed as described in Callahan et al. (Callahan et al., F1000Research 5, 1492 (2016)), which includes using Phyloseq (McMurdie, P. J. and Holmes, S., PLOS One 8, e61217 (2013)). Taxa that were present less than 5 times in total and present in less than 5% of the samples were removed. qPCR data was integrated with the abundance data using the script provided in Vandeputte et al. (Vandeputte, D. et al., Nature 551, 507-511 (2017)). Wilcoxon signed-rank test and Benjamini-Hochberg correction were performed to compare individual ASVs in different treatments and ASVs with an adjusted p-value of less than 0.1 were considered as statistically significant. We compared the biofilm communities exposed to jmt rVOCs and jmt rVOCs+MeJA to get MeJA responders (FIG. 4A, 4C). Similarly, rVOCs responders (FIG. 4B) were identified by comparing communities exposed to soil VOCs and WT rVOCs. The phylogenetic tree was constructed using Phangorn (Schliep, K. P., Bioinformatics 27, 592-593 (2011).) package and visualized using iTOL (Letunic, I. & Bork, P., Nucleic Acids Res. 49, W293-W296 (2021)).

PICRUSt 2.0 pipeline was used to understand the predicted functions of the community. The differential functions were identified in the same way as identification of differential taxa (integration with qPCR bacterial load data with gene tables followed by Wilcoxon Rank Sum test with Bonferroni-Hochberg correction).

Effects of Complex Biofilms on Hosts from a Distance

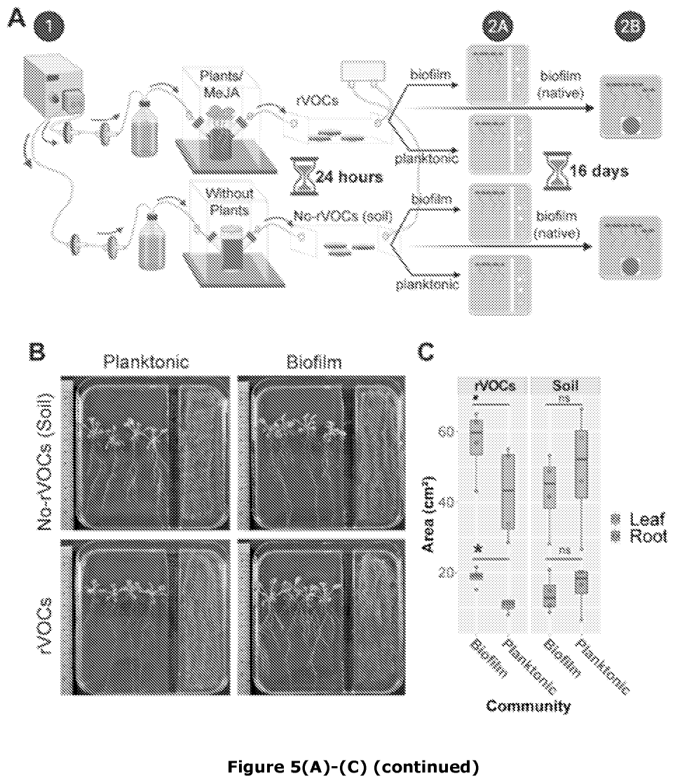

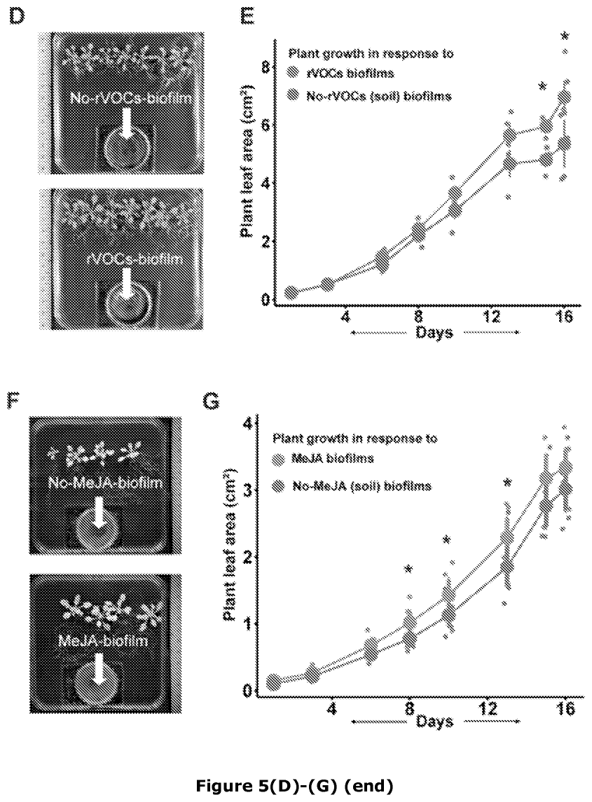

The host benefit assay system of biofilms consists of two major parts (FIG. 5A). 1. Induction of biofilm with and without rVOCs/MeJA using the “push-pull” system; and 2. Monitoring the growth of plants exposed to volatiles from induced biofilms (FIG. 5A). 2 ml of Microbial inoculum was taken in a small Petri plate (35 mm) and exposed to root VOCs and soil VOCs over 24 hours using “push-pull dynamic” system to generate rVOCs and No-rVOCs-induced biofilms respectively. After that, the planktonic fraction was gently taken out to separate both planktonic and biofilm phases of the soil inoculum of each treatment.

For assaying the plant response with intact biofilm as depicted in “2B” of FIG. 5A, 500 μl of fresh SEM liquid medium was added to the biofilms. 50 ml of SEM-agar (1%) was poured in the square plates (120×120 mm) to prepare the plates with medium to perform the plant response assay. The part of the medium was scraped off to create the space for small Petri plates with intact biofilms. The small Petri plates with biofilms were then placed with 4 days old axenic Arabidopsis seedlings in shared headspace for co-culturing into the growth room with the control environment. The non-destructive images were taken at regular intervals to study the growth dynamics. The leaf area was calculated using an ImageJ macro. The statistical modelling of the data was performed using linear mixed effects models (Table 3 and Table 4).

Isolation, Biofilm Assay of Monoculture Strains, and their Effect on Plant Growth

Complex microbiota biofilm exposed to volatile MeJA was scraped off and resuspended in PBS. Through serial dilution, this inoculum was plated on soil extract agar, minimal media agar, and LB agar. Colonies with unique morphology were picked and streaked onto a fresh LB plate to acquire single colonies. The isolated strains were identified using Sanger sequencing of the PCR product with primers 27F and 11.

Their biofilm response to volatile MeJA was tested using “push-pull” setup (as described above for complex biofilms). Their response to soluble MeJA was tested by directly adding MeJA to the monoculture inoculum (final concentration of 5 nM). For both assays, biofilms were stained with crystal violet and quantified after 24 hours as explained in the biofilm staining protocol. The initial OD of the inoculum was 0.2.

To test the effect of isolated strains on the plant growth from a distance, bipartite assay was performed where 50 ul of 0.2 OD inoculum was smeared on part of the plate and 3-5 seedlings (four days old) were placed in the other part of the plate without spatial contact. Plant growth was monitored non-invasively using photography. Leaf area was quantified using an ImageJ macro as described in the previous section.

### Example 1

Plant Root VOCs Promote Biofilm Formation in the Soil Microbial Community

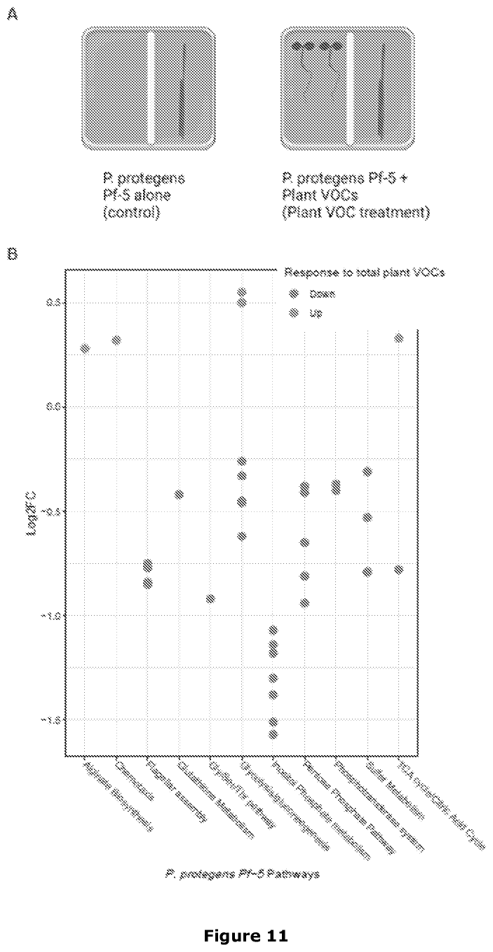

To understand the effects of plant VOCs on PGPR, a known model PGPR, Pseudomonas protegens Pf-5, was co-cultured with Arabidopsis seedlings in vitro with shared headspace without physical contact to ensure only gaseous interaction (FIG. 11A). Transcriptome profiling of Pseudomonas protegens Pf-5 with and without plant VOCs was then performed 3 days post-inoculation. It was observed that Pf-5 exposed to plant VOCs showed overall repression of metabolic pathway and flagellar motility-related genes (FIG. 11B). At the same time, it was also observed that there was a significant induction of certain biofilm-related genes as compared to P. protegens Pf-5 without plant VOCs (FIG. 11B). Such reprogramming of metabolic and motility-related genes is suggestive of a biofilm lifestyle, and that plant VOCs promote biofilm formation even in complex soil microbiome.

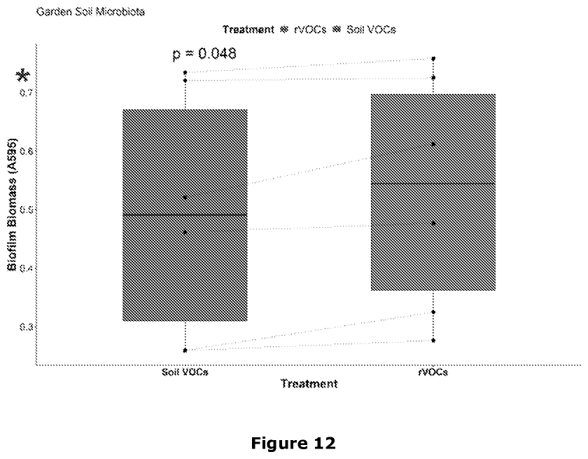

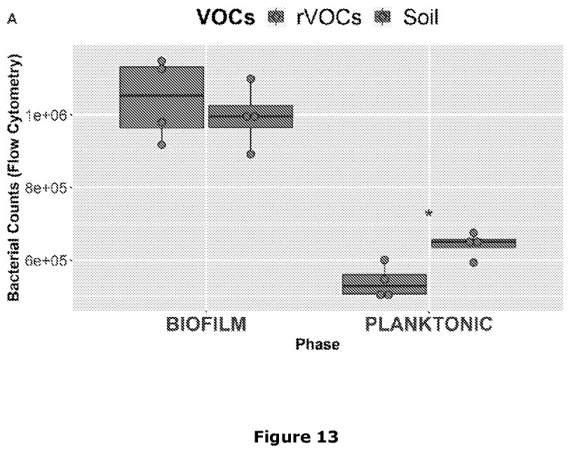

To test the effect of total plant VOCs on the soil microbiome community (FIG. 1A), soil microbiome suspension was first exposed to VOCs from 14-days old Arabidopsis seedlings in a static headspace plate assay system (FIG. 1B, methods). At 24 hours, microbiota exposed to plant VOCs showed significantly higher biofilm biomass as compared to control without plants (FIG. 1C). Next, in order to test whether the source of these biofilm-inducing VOCs was from the roots (referred here as rVOCs), a modular setup termed as “Push-pull airflow dynamic system” (FIG. 1D, Methods) was designed. Using this setup, sterile and humid airflow was directed through plant roots towards the inoculum of the soil microbiome. Consistent with the phenomenon observed in the “static system” assay, microbiota exposed to rVOCs showed significantly higher biofilm biomass at 40 hours (FIG. 1E). rVOC-induced biofilms were formed independently of soil types (FIG. 12). Lower bacterial counts in the planktonic phase and higher bacterial count in the biofilm phase exposed to rVOCs (FIG. 13) also confirmed that it is not a universal growth promotion of bacteria but shift of microbes from planktonic to biofilm phase.

To further determine the universality of the observed phenomenon, induction of biofilms by rVOCs from different plant species spread across the plant kingdom (pteridophyte, monocots, and dicots) that are separated by at least 400 million years of evolution was tested. The dynamic push-pull system was chosen due to its advantage in testing rVOCs activity from soil-grown roots. In all cases, rVOCs from these diverse plant species promoted biofilm formation in soil microbiota (FIG. 1F).

### Example 2

Methyl jasmonate is a potent rVOC that signals biofilm promotion in the soil microbiome

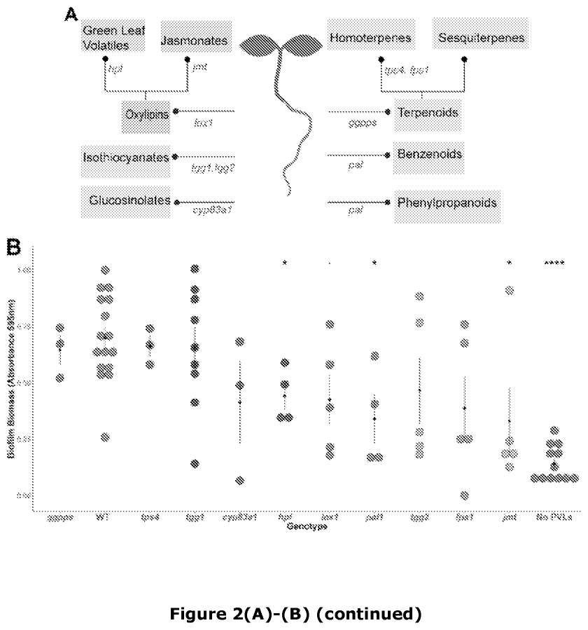

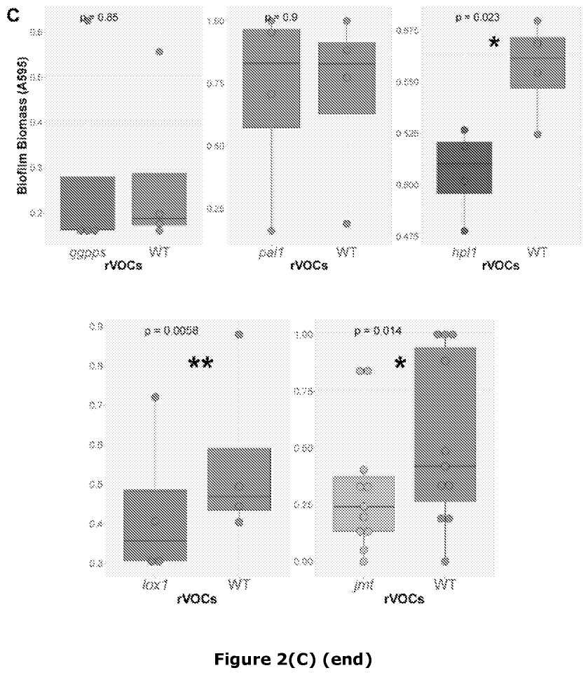

To identify the class(s) of plant rVOCs responsible for triggering the biofilm formation in soil microbiome, a genetic approach was taken that involved screening biosynthetic mutant lines of Arabidopsis for the major known classes of plant VOCs. Of the 10 mutant lines belonging to the biosynthesis of 6 VOC classes namely terpenoids, benzenoids, phenylpropanoids, glucosinolates, and oxylipins (FIG. 2A), four mutant lines that belonged to the oxylipin biosynthetic pathway and phenylpropanoid pathway (lox1, hpl1, jmt, pal1) produced significantly less biofilms compared to the WT control in the static assay system (FIG. 2B). These four mutants were tested along with ggpps in the dynamic push-pull system for the ability of their rVOCs to induce biofilms. In this assay, the oxylipin mutants (hpl1, jmt, lox1) were unable to promote biofilms compared to their respective WT control (FIG. 2C). These results, taken together, indicate the possible role of oxylipins as the bioactive components of the rVOCs in promoting biofilms in the soil microbiome.

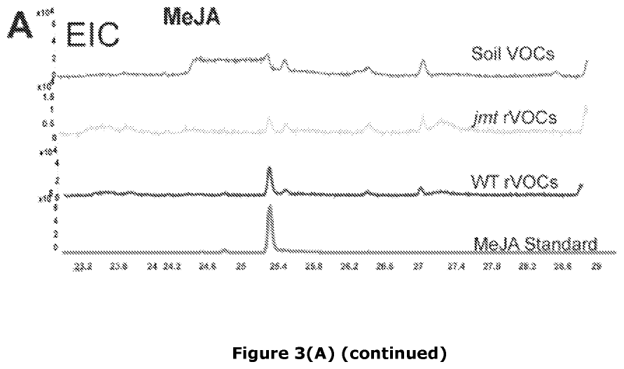

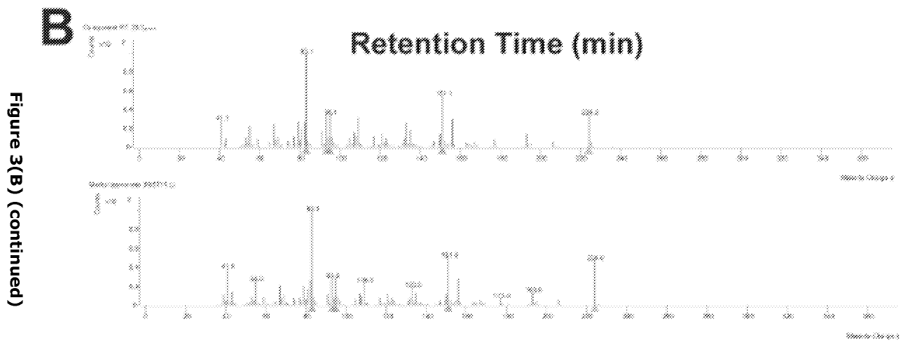

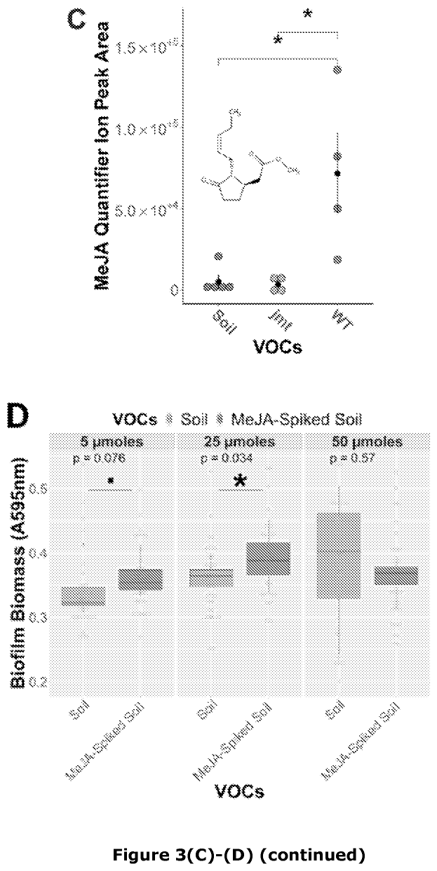

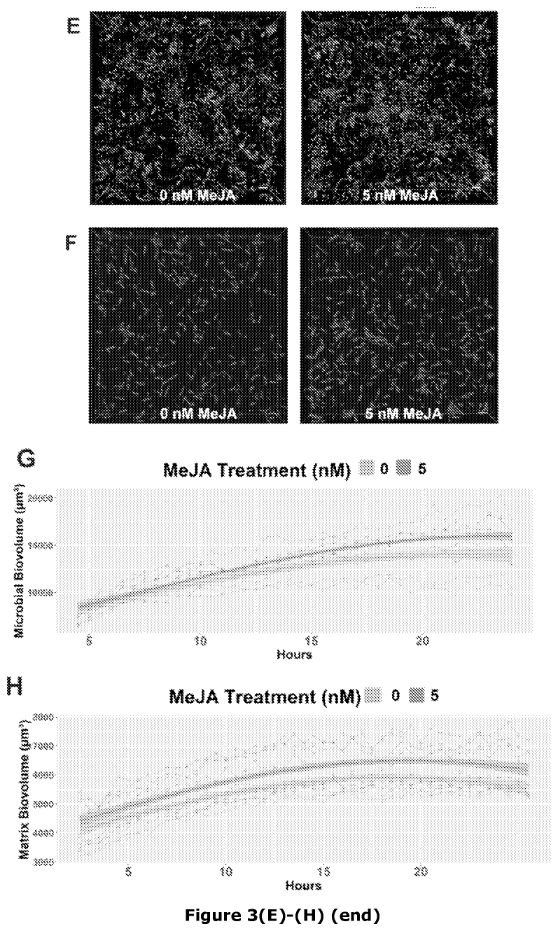

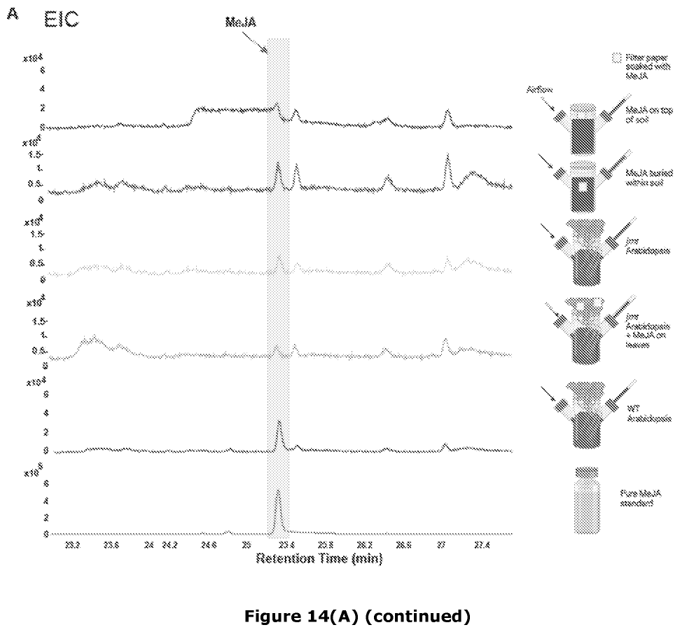

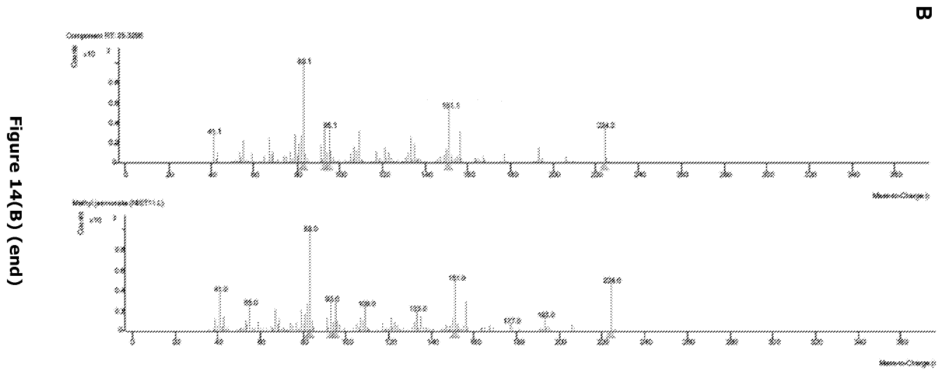

Methyl jasmonate (MeJA) is known to be one of the major bioactive compounds in the oxylipin class of plant volatiles. Given the involvement of LOX1 and JMT genes in MeJA biosynthesis and the inability of their mutants to promote biofilms, we tested whether the plant roots release MeJA as a VOC. The presence of MeJA was detected in the rVOCs of Arabidopsis, using a polymer-packed cartridge followed by direct thermal desorption (FIG. 3A, B). MeJA levels were significantly higher in WT Arabidopsis seedlings than both soil and jmt mutants in the dynamic system thus showing the MeJA detected is of plant origin (FIG. 3C). To test whether the origin of MeJA was from roots or shoots, pieces of filter paper soaked with MeJA were placed above and within soil along with jmt mutants (FIG. 14A-B). The levels of MeJA detected in the rVOCs from filters placed on leaves of jmt mutants were comparable to those of jmt without the filters or the soil alone, thereby establishing that the MeJA was released from the belowground portion of the plants.

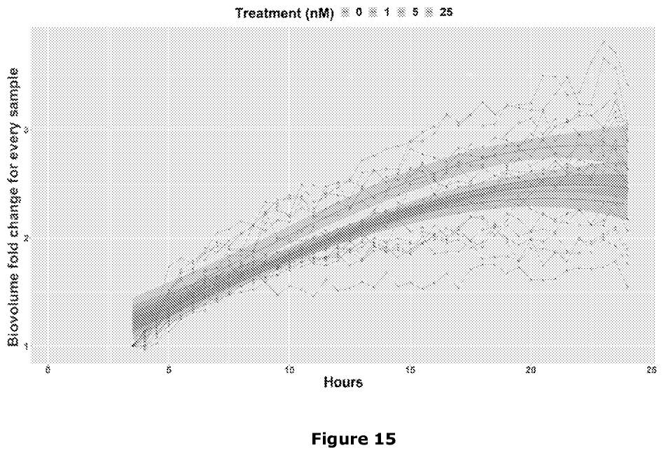

As MeJA can exist in both soluble and volatile forms, both forms were tested for their biofilm-promoting activity. To test the effect of volatile form, 5, 25, and 50 μmol of MeJA was spiked in the soil chamber of the dynamic system setup followed by quantification of the biofilm in the recipient chamber. The potency of MeJA in biofilm promotion is higher at low concentrations (5 μmol) and gradually declined in a dose-dependent manner (FIG. 3D). To test the effect of soluble form and to study the spatio-temporal dynamics of MeJA in promoting biofilms, the biovolumes of soil microbiome and matrix was quantified in both time-dependent (0 to 24 hours) and MeJA concentration-dependent (0, 1, 5, 25 nM) manner using live confocal imaging (FIG. 2E-F, FIG. 15). Microbiota treated with 5 nM MeJA showed an increasingly higher biofilm growth trajectory compared to the control (FIG. 2G). Compared to the non-treated biofilms, the interaction of time and 5 nM treatment was statistically significant as shown by mixed-effects model (FIG. 2G, Table 1). Similarly, 5 nM MeJA also promoted biofilm matrix compared to non-treated biofilm (FIG. 2H, Table 2). MeJA influence appeared earlier in the matrix starting from 7 hour onwards compared to biovolumes that differed from 15 hours onwards (FIG. 3G-H). The overall results validated that both liquid and volatile forms of MeJA can promote biofilm formation in soil microbiota in a dose-dependent manner. The results also revealed that MeJA affects both the members and matrix to modulate the biofilm growth dynamics.

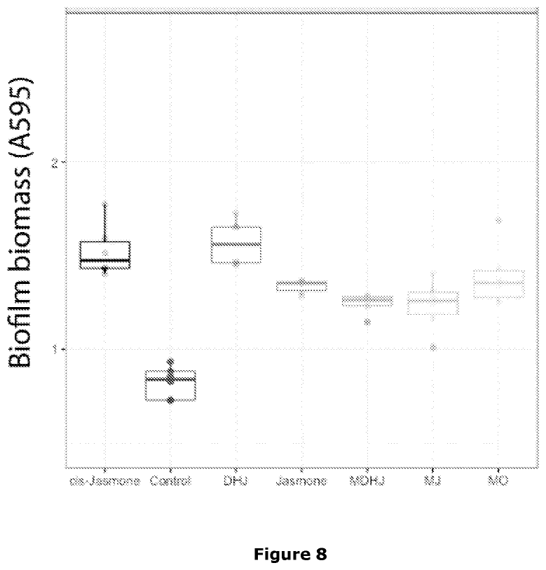

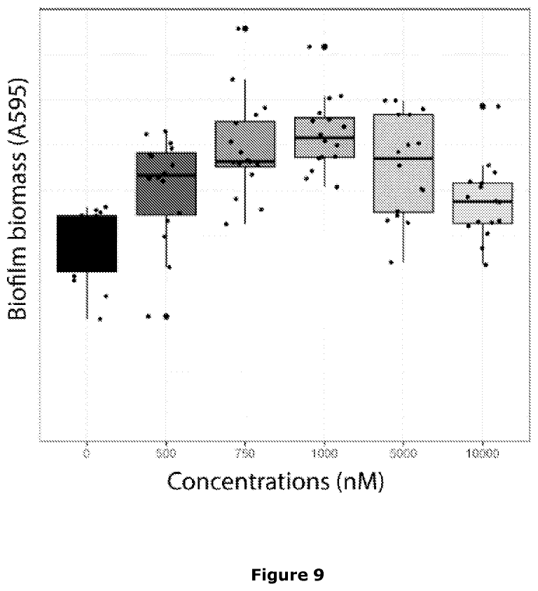

To identify the potential functional group of MeJA for its biofilm-inducing property, several jasmonic acid analogues were screened for their biofilm-inducing capability in the soil microbiome. The analogues were used to study biofilm induction in both complex soil microbiomes (FIG. 8). Cis-jasmone and dihydro-jasmonic acid (DHJ) had the highest potential for biofilm induction.

Biofilm induction of the soil microbiome was tested with several concentrations of pure MeJA at the nanomolar level. It was confirmed that a concentration range from 500 nM of MeJA was effective to induce biofilms without being detrimental to plant growth.

TABLE 1
Statistical Modeling of Live Imaging (Syto9: Microbiome)

| Ime((Biovolume)~ | | | | | |
|---|---|---|---|---|---|
| Hours*Treatment + | | | | | |
| I(Hours\hat{ }2), random = | | | | | |
| ~1 \| Position, # Random | | | | | |
| Effect is replicates | | | | | |
| correlation = corAR1( ), | | | | | |
| #Temporal Correlation— | | | | | |
| Auto Regressive data = mj, | | | | | |
| method = “ML”, control = | | | | | |
| ImeControl(maxIter = 500, | | | | | |
| msMaxIter = 500)) | Value | Std. Error | DF | t-value | p-value |
| (Intercept) | 3327.92 | 1115.548 | 697 | 2.983215 | 0.002952 |
| Hours | 1050.706 | 98.80455 | 697 | 10.63419 | 1.40E−24 |
| Treatment1 nM | −988.35 | 1508.955 | 14 | −0.65499 | 0.523091 |
| Treatment25 nM | −1245.62 | 1422.656 | 14 | −0.87556 | 0.396042 |
| Treatment5 nM | −453.513 | 1508.955 | 14 | −0.30055 | 0.768176 |
| I(Hours\hat{ }2) | −26.3094 | 3.157457 | 697 | −8.33247 | 4.20E−16 |
| Hours: Treatment1 nM | 106.209 | 61.19894 | 697 | 1.735471 | 0.083099 |
| Hours: Treatment25 nM | 90.7811 | 57.69891 | 697 | 1.573359 | 0.11609 |
| Hours: Treatment5 nM | 102.9409 | 61.19894 | 697 | 1.682071 | 0.093003 |

TABLE 2
Statistical Modeling of Live Imaging (SYPRO: Biofilm Matrix)

| model <- | | | | | |
|---|---|---|---|---|---|
| Ime((Biovolume)~ | | | | | |
| Hours*Treatment + | | | | | |
| I(Hours\hat{ }2), random = | | | | | |
| ~1 \| Position, # Random | | | | | |
| Effect is replicates | | | | | |
| correlation = corAR1( ), | | | | | |
| # Temporal Correlation— | | | | | |
| Auto Regressive | | | | | |
| data = mj2, method = | | | | | |
| “ML”, control = | | | | | |
| ImeControl(maxIter = | | | | | |
| 500, msMaxIter = 500), | | | | | |
| na.action = na.exclude) | Value | Std. Error | DF | t-value | p-value |
| (Intercept) | 3364.706 | 254.3009 | 528 | 13.2312 | 1.02E−34 |
| Hours | 274.4583 | 10.76781 | 528 | 25.48878 | 4.93E−94 |
| Treatment5 | 294.3537 | 351.7046 | 10 | 0.836934 | 0.42218 |
| I(Hours\hat{ }2) | −7.27922 | 0.368953 | 528 | −19.7294 | 2.54E−65 |
| Hours: Treatment5 | 11.24781 | 4.744255 | 528 | 2.370826 | 0.018106 |

TABLE 3
Plant growth in response to rVOC-induced biofilms

| Ime(log(Area . . . cm2.)~ | | | | | |
|---|---|---|---|---|---|
| Treatments * Days + | | | | | |
| I(Days\hat{ }2), | | | | | |
| random = ~1 \| | | | | | |
| Replicates/Treatments, | | | | | |
| data = dat1, method = | | | | | |
| “ML”, control = | | | | | |
| (msMaxIter = 100)) | Value | Std. Error | DF | t-value | p-value |
| (Intercept) | −1.91547 | 0.067698 | 53 | −28.2945 | 1.17E−33 |
| Treatments—rVOCs | −0.01347 | 0.082845 | 3 | −0.16265 | 0.881132986 |
| Days | 0.443531 | 0.012742 | 53 | 34.80913 | 3.45E−38 |
| I(Days\hat{ }2) | −0.01393 | 0.000694 | 53 | −20.0697 | 1.99E−26 |
| Treatments—rVOCs: | 0.017789 | 0.00582 | 53 | 3.056589 | 0.003502745 |
| Days | | | | | |

Contrasts

| | | | | | % change in |
|---|---|---|---|---|---|
| | | | | | leaf area |
| | | | | | (compared to |
| | | | | | soil-VOC |
| Days | SE | df | t. ratio | p. value | Biofilms) |
| 1 | 0.08 | 3 | 0.054 | 0.96 | 0.40% |
| 3 | 0.076 | 3 | 0.546 | 0.623 | 4.10% |
| 6 | 0.073 | 3 | 1.402 | 0.255 | 9.80% |
| 8 | 0.073 | 3 | 1.999 | 0.139 | 13.80% |
| 10 | 0.076 | 3 | 2.551 | 0.084 | 17.90% |
| 13 | 0.085 | 3 | 3.19 | 0.05 | 24.30% |
| 15 | 0.094 | 3 | 3.467 | 0.04 | 28.80% |
| 16 | 0.1 | 3 | 3.567 | 0.038 | 31.10% |

TABLE 4
Plant growth in response to MJ-induced biofilms

| Ime(log(Avg_leaf_area)~ | | | | | |
|---|---|---|---|---|---|
| Treatment * Day + | | | | | |
| I(Day\hat{ }2), random = ~1 \| | | | | | |
| Replicate/Treatment, | | | | | |
| data = mj2, method = | | | | | |
| “ML”, control = | | | | | |
| (msMaxIter = 100)) | Value | Std.Error | DF | t-value | p-value |
| (Intercept) | −2.58433 | 0.118413 | 53 | −21.8248 | 3.75E−28 |
| Treatment—MeJA- | 0.314091 | 0.065779 | 3 | 4.774965 | 0.017455 |
| biofilm | | | | | |
| Day | 0.350382 | 0.01389 | 53 | 25.22577 | 3.38E−31 |
| I(Day\hat{ }2) | −0.00767 | 0.000756 | 53 | −10.1354 | 5.25E−14 |
| Treatment—MeJA- | −0.01093 | 0.006344 | 53 | −1.72356 | 0.090617 |
| biofilm: Day | | | | | |

Contrasts

| | | | | | percent. |
|---|---|---|---|---|---|
| Day | SE | df | t. ratio | p. value | change |
| 1 | 0.082 | 3 | 5.023 | 0.015 | 35.40% |
| 3 | 0.066 | 3 | 5.608 | 0.011 | 32.50% |
| 6 | 0.048 | 3 | 6.574 | 0.007 | 28.20% |
| 8 | 0.042 | 3 | 6.811 | 0.006 | 25.40% |
| 10 | 0.041 | 3 | 6.154 | 0.009 | 22.70% |
| 13 | 0.049 | 3 | 4.157 | 0.025 | 18.80% |
| 15 | 0.058 | 3 | 2.992 | 0.058 | 16.20% |
| 16 | 0.063 | 3 | 2.524 | 0.086 | 14.90% |

### Example 3

Phylogenetically Diverse Taxa from the Soil Microbiota Biofilms Respond to rVOCs and MeJA

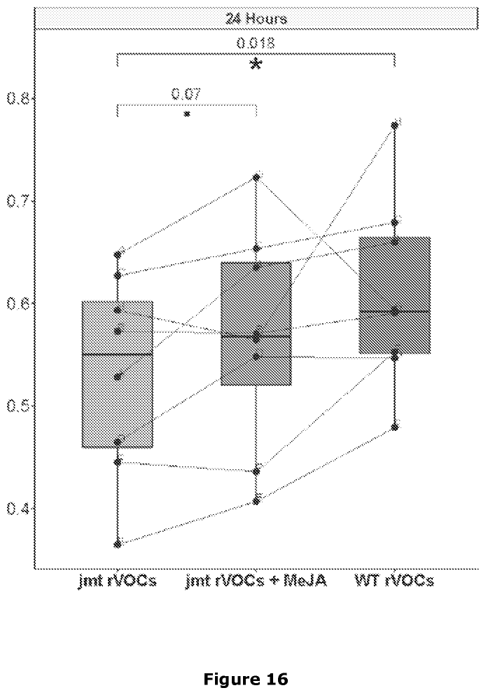

Three lines of evidence, namely 1) compromised ability to induce biofilms by jmt mutant; 2) release of volatile MeJA from plant roots; and 3) biofilm induction by pure MeJA, taken together indicate that root-derived volatile MeJA promoted biofilms in soil microbiota. This was further corroborated by the comparable biofilm induction by WT rVOCs and rVOCs from jmt mutant complemented with pure MeJA (imt+MeJA) around the rhizosphere (FIG. 4A, FIG. 16A). The taxonomically diverse members of the soil microbiome was next investigated for their ability to respond to WT rVOCs and MeJA using 16S rRNA gene amplicon sequencing (FIG. 4A).

Alpha diversity analysis revealed that biofilm phase hosted a significantly higher diversity as compared to the planktonic phase across all the samples (FIG. 4B, FIG. 17C). All biofilm communities exposed to different VOC treatments had comparable diversity as shown by Shannon indices (Table 5). After prevalence filtering, it was observed that the inoculum consisted of 1241 Amplicon Sequence Variants (ASVs) that differentiated into biofilm (1039 ASVs) and planktonic (962 ASVs) mode of lifestyle with a high degree of overlap. Across the biofilm and planktonic samples, different VOC treatments explained only 2-6% of the total variance (FIG. 17E) implying that there is no major compositional turnover of the community in a timeframe of 24 hours. However, this did not exclude the possibility of small shifts in the abundances of specific strains in the community.

To obtain absolute abundance of each ASV in the biofilm community, the bacterial load of each sample (quantified through through qPCR) was integrated with the microbiome profile. To identify the biofilm members responding to WT rVOCs, soil VOC-induced and WT rVOCs-induced biofilm communities were compared. Similarly, MeJA-responding biofilm members were identified by comparing jmt rVOCs-induced and jmt+MeJA-induced biofilm communities.

It was observed that the WT rVOCs induced a significant shift in the abundance of ˜8% (86 ASVs inclusive of 24 promoted and 62 repressed ASVs) of the biofilm community compared to those exposed to soil VOCs (FIG. 4C). Interestingly, the responding subcommunity was different between 16 and 24 hours. MeJA also induced a significant shift in the abundance of ˜10% (103 ASVs inclusive of 21 promoted and 82 repressed) of the biofilm community (FIG. 4D). Like rVOC-responders, MeJA responders varied between 16 and 24 hours indicating rapid community succession. The community shift after 16 hours corroborates the shift in biovolume in similar time in response to MeJA (FIG. 3G).

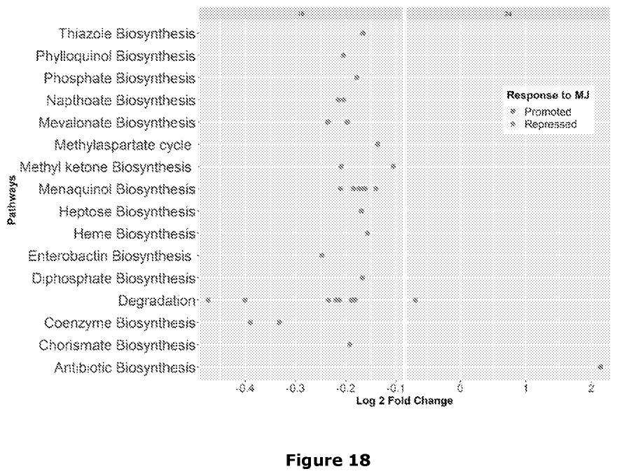

The responder communities, at both 16 and 24 hours, consisted mostly of repressed taxa as compared to the induced taxa (FIG. 4C-D). In response to MeJA treatment for 16 hours, there was an overall repression of metabolic biosynthetic pathways based on their predicted functions, which is consistent with lifestyle shift from planktonic to biofilm mode (FIG. 11B and 18). Interestingly, this repression was lifted after the community shift happens at 24 hours (FIG. 18).

Overall, rVOCs and MeJA dynamically induced subtle changes in phylogenetically diverse strains within the soil microbiome biofilms.

TABLE 5
Diversity indices of biofilm communities exposed to
different VOCs.

| Biofilm Community | Richness | Evenness |
|---|---|---|
| Soil VOC-exposed | 762 | 0.614 |
| Jmt rVOCs-exposed | 772 | 0.607 |
| WT rVOCs-exposed | 753 | 0.5963 |
| Jmt rVOCs + MeJA-exposed | 736 | 0.583 |

### Example 4

rVOCs-Induced Complex Biofilms Promote Plant Growth

Given the evolutionary conserved nature of the bioactivity of rVOCs on soil biofilms, it was hypothesised that there would be a reciprocal ecological role of these complex biofilms on plant growth possibly through volatile signals. In order to simulate the long-distance effect of MeJA-induced biofilms, their benefit to the host plants was tested from a distance. Functional in vitro assays of plant growth were conducted with planktonic and biofilm microbiota, as well as with intact complex biofilms (FIG. 5A). After two weeks of co-culturing, plants associated with disrupted rVOCs-induced-biofilm fraction appeared healthy with a significantly higher root and leaf area relative to rVOCs-planktonic community (FIG. 5B, C). However, soil-VOC induced planktonic and biofilm fractions did not lead to a significant difference in plant biomass. This indicated that in the presence of plant signals, biofilm communities provide more plant growth benefits than the planktonic communities. In contrast, in the absence of plant signals, biofilm and planktonic communities provided comparable growth benefits. Hence, plant signals shift the host benefits through a biofilm-preferred mode. Next, to investigate whether undisrupted biofilms recapitulate the plant growth promotion, the effect of rVOC-biofilms in their native form on plant growth dynamics was studied over two weeks. The rVOC-biofilms promoted plant growth with an increasing benefit from as early as 6 days and led to significant differences from 13 days onwards, compared to the soil-VOC biofilms (FIG. 5D, E). Given the biofilm-promoting role of MeJA, the functionality of pure MeJA-promoted undisrupted biofilms on plant growth was also tested. Interestingly, the pure MeJA-induced biofilms also led to significantly higher leaf area compared to soil VOC-induced biofilm from day 8 to day 13 (FIG. 5F, G).

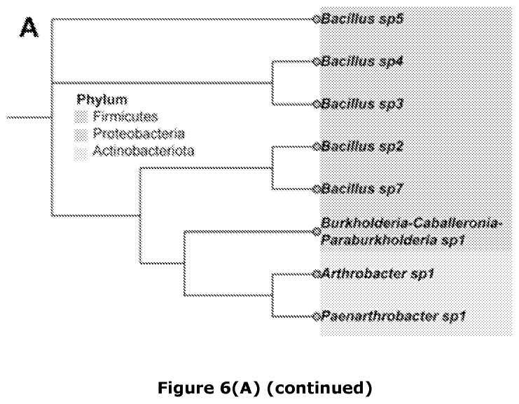

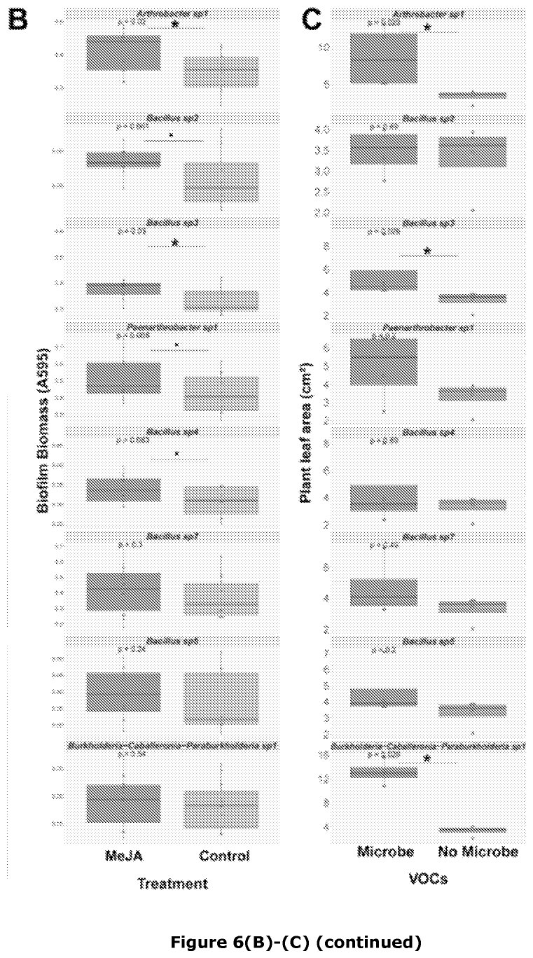

Plant growth promotion by complex biofilms was next tested to investigate whether these traits can be recapitulated at an individual strain-level. Randomly-selected strains were cultured from MeJA-induced complex biofilms (FIG. 6A). Next, the MeJA-responsiveness to form biofilms and plant growth promotion trait from a distance was tested using the same set-up as for complex biofilms and microbiota (FIG. 5A). Three out of nine strains tested, led to an increase in total plant leaf area by 25-300%, indicating plant growth promotion (FIG. 6B-C), likely involving microbial VOCs. Of the nine strains, six were MeJA-responsive in biofilm formation (FIG. 6D). Interestingly, two out three of the plant growth promoting strains were also MeJA-responsive. Some of the MeJA-responsive strains did not have the plant growth promoting effect.

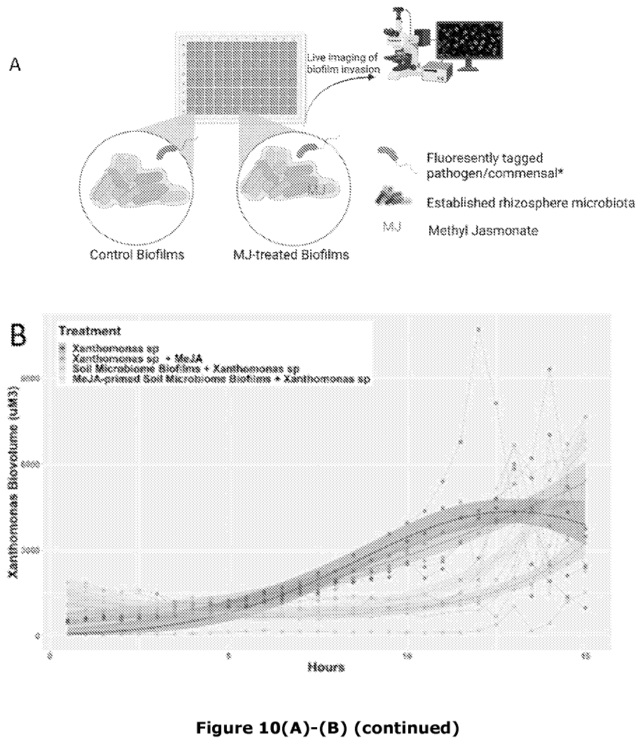

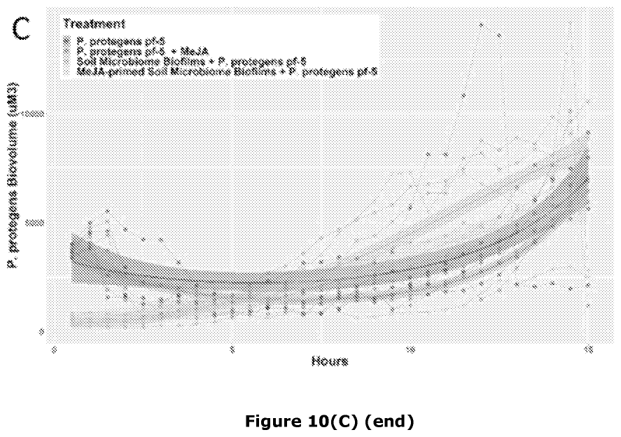

The ability of MeJA-induced biofilms to get invaded by a plant pathogen (Xanthomonas sp) and a plant-beneficial strain (P. protegens Pf-5) was tested. The tested pathogen Xanthomonas sp is closely related to wilt causing Xanthomonas campestris that affects a wide variety of cruciferous vegetables worldwide. MeJA-induced biofilms trapped a significantly higher number of plant pathogens (FIG. 10B) and a lower number of plant-beneficial strains (FIG. 10C). This implied that MeJA-induced biofilms formed an outer protective boundary in the soil to trap pathogens and only allow beneficial strains to enter the rhizosphere.

### Example 5

Isolation of Plant Beneficial Isolates from Soil Biofilms Using MeJA

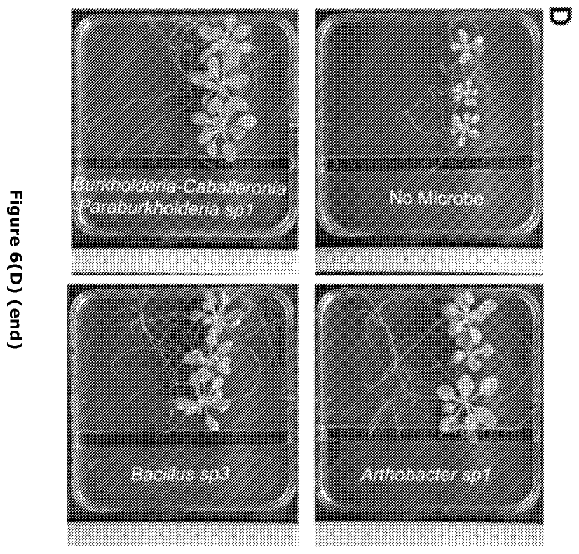

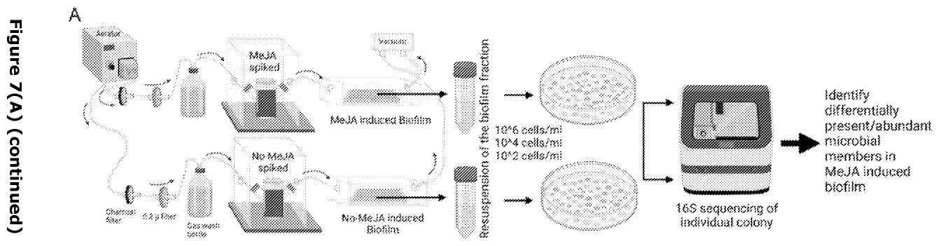

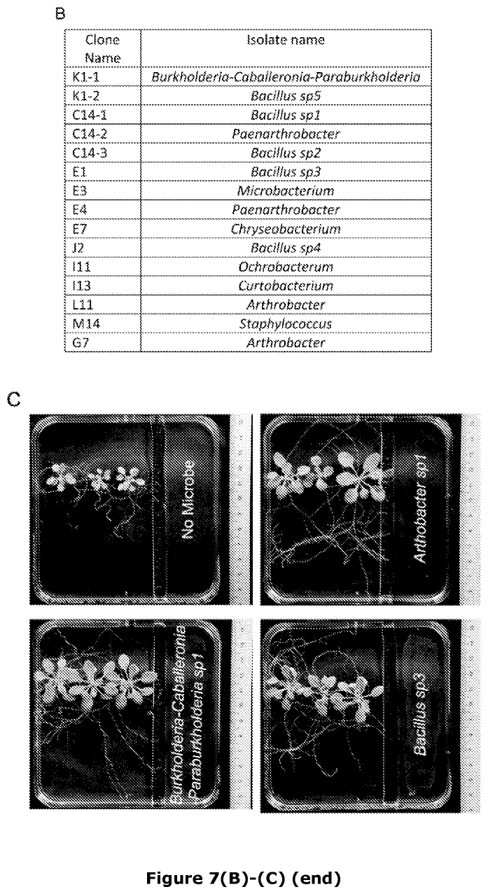

MeJA-responders were isolated to study their effects on plants. A novel workflow was developed for quick isolation of the MeJA responders through a combination of the push-pull system and a culturomics approach (FIG. 7A). In this workflow, MeJA-induced biofilms were generated through the push-pull system and the biofilm fraction was resuspended in the soil extract medium (SEM) at various dilutions. Next, the suspension was spread in a different medium (LB, M9, SEM) supplemented with MeJA. The MeJA-supplemented medium was compared to the non-supplemented medium plates, and unique and differentially abundant colonies were selected. The isolates were identified through Sanger sequencing of 16S rRNA genes of selected colonies. Adopting this workflow, 15 MeJA-responding members were isolated from the soil microbial biofilms (FIG. 7B) and studied their plant growth promoting response in the in vitro plate assay system. Some of these isolates provided significant growth promotion to plants (FIG. 7C).

1. A method for promoting plant growth in a growth medium, the method comprising inducing biofilm growth in the growth medium by providing an oxylipin to the growth medium, wherein induction of biofilm growth promotes plant growth in the growth medium.

2. The method according to claim 1, wherein the oxylipin is a jasmonate or a derivative thereof.

3. The method according to claim 1 or 2, wherein the oxylipin is provided as a liquid.

4. The method according to any one of claims 1 to 3, further comprising a step of enriching the growth medium with plant growth microbes.

5. The method according to any one of claims 1 to 4, wherein the plant is a species selected from bryophyte, pteridophyte, gymnosperm, monocot, and dicot.

6. The method according to claim 5, wherein the plant is a cultivated monocot or dicot.

7. The method according to any one of claims 1 to 6, wherein the growth medium is soil.

8. A method for inducing biofilm growth in a growth medium, the method comprising providing an oxylipin to the growth medium, wherein the oxylipin induces biofilm growth in the growth medium.

9. The method according to claim 8, wherein the oxylipin is a jasmonate or a derivative thereof.

10. The method according to claim 9, wherein the method is capable of promoting plant growth in a growth medium.

11. The method according to any one of claims 8 to 10, wherein the biofilm is capable of sequestering a plant pathogen.

12. A growth medium for promoting plant growth, wherein the growth medium comprises an oxylipin.

13. A kit for promoting plant growth, wherein the kit comprises a growth medium, wherein the growth medium comprises an oxylipin.

14. A method for isolating a microbe that is responsive to an oxylipin, the method comprising culturing a plurality of microbes in a growth medium comprising the oxylipin, and isolating a microbe that is enriched in the growth medium as compared to a growth medium that does not contain the oxylipin.

15. The method for claim 14, wherein the method comprises identifying the isolated microbe.
